(19) United States
(12) Patent Application Publication
(10) Pub. No.: US 2024/0245756 A1
(43) Pub. Date: Jul. 25, 2024

(54) CHIMERIC PROTEINS AND METHODS OF USE FOR TREATMENT OF NEURODEGENERATIVE DISORDERS

(71) Applicant: Silver Creek Pharmaceuticals, Inc., San Francisco, CA (US)

(72) Inventors: Kristopher M. Kuchenbecker, Phoenix, AZ (US); Samuel J. Pfaff, Wyncote, PA (US); Walter Olsen, Dracut, MA (US); Lakhmir S. Chawla, San Diego, CA (US)

(73) Assignee: Silver Creek Pharmaceuticals, Inc., San Francisco, CA (US)

(21) Appl. No.: 18/417,568

(22) Filed: Jan. 19, 2024

Related U.S. Application Data
(60) Provisional application No. 63/480,547, filed on Jan. 19, 2023.
(60) Provisional application No. 63/597,975, filed on Nov. 10, 2023.

(51) Int. Cl. A61K 38/30; A61K 38/17; A61K 38/38; A61P 25/16; A61P 25/28

(52) U.S. Cl. CPC A61K 38/30; A61K 38/1709; A61K 38/385; A61P 25/16; A61P 25/28

(57) ABSTRACT

Aspects of the present disclosure relate generally to methods for treating neurodegenerative disorders with chimeric proteins and pharmaceutical compositions comprising such chimeric proteins.

### RELATED APPLICATIONS

This application claims priority to and the benefit of U.S. Provisional Application No. 63/480,547, filed Jan. 19, 2023, and of U.S. Provisional Application No. 63/597,975, filed Nov. 10, 2023, the contents of each of which are incorporated herein by reference in their entireties.

### REFERENCE TO SEQUENCE LISTING

This specification includes a sequence listing submitted herewith, which includes the file entitled 132463-011102.xml having the following size: 38,100 bytes which was created Jan. 16, 2024, the contents of which are incorporated by reference herein.

### TECHNICAL FIELD

Aspects of the present disclosure relates generally to chimeric proteins and pharmaceutical compositions comprising such chimeric proteins, and methods for using such chimeric proteins for treating neurodegenerative disorders in a subject in need thereof.

### BACKGROUND

Brain injuries and central nervous system disorders are common medical condition that can result in disabling and irreversible degradation of a person's cognitive and sensorimotor capacity. Brain injuries and central nervous system disorders that can result in nerve cell death and damage range from ischemic stroke to degenerative disorders.

Neurodegenerative diseases such as Parkinson's disease and other alpha-synucleinopathies are chronic debilitating disorders. Thus there remains a need for new treatments to treat or slow the disease progression.

### SUMMARY

Methods for treating neurodegenerative disorders are provided. In some embodiments, the neurodegenerative disorder is a synucleinopathy. In some embodiments, the neurodegenerative disorder is Parkinson disease or Alzheimer disease.

In some embodiments, the method comprises administering to a subject in need thereof a pharmaceutical composition comprising a therapeutically effective amount of a chimeric protein and a pharmaceutically acceptable carrier, wherein the chimeric protein comprises (a) a targeting domain comprising a variant of human Annexin 5 (AnxV) comprising one or more mutations, wherein the one or more mutations consist of a substitution at the position corresponding to C316 and optionally at one or more positions corresponding to R63, K70, K101, E138, D139, N160, and combinations thereof; (b) an activator domain comprising a variant of human insulin-like growth factor IGF-1 comprising one or more mutations, wherein the one or more mutations consist of a substitution at one or more positions corresponding to E3, Y24, Y31, Y60, and combinations thereof, wherein the variant of IGF-1 decreases activation of the IGF-1 receptor relative to the wild-type IGF-1, and (c) a half-life modulator comprising a variant of human serum albumin (HSA) comprising one or more mutations, wherein the one or more mutations consist of a substitution at one or more positions corresponding to C58 and N527, and combinations thereof.

In some embodiments, the administration of the pharmaceutical composition results in targeted stimulation of the phosphorylation of serine/threonine protein kinase B (AKT) pathway by selective activation of the IGF-1 receptor. In some embodiments, the administration of the pharmaceutical composition results in reduction or prevention of amyloid fibril formation.

In some embodiments, the pharmaceutical composition is administered intrathecally. In some embodiments, the pharmaceutical composition is administered intravenously.

In some embodiments, the variant of IGF-1 decreases activation of the IGF-1 receptor relative to the wild-type IGF-1. In some embodiments, the variant of IGF-1 comprises E3R and Y31A substitutions relative to wild type human IGF-1.

In some embodiments, the variant of human Annexin 5 comprises the amino acids 2-320 corresponding to wild type human Annexin 5 and comprises R63A, K70A, K101A, E138A, D139G, N160A, C316A substitutions relative to wild type human Annexin 5.

In some embodiments, the variant of human serum albumin comprises the amino acids 25-609 corresponding to wild type human serum albumin and comprises C58S and N527Q substitutions relative to wild type human serum albumin.

In some embodiments, the chimeric protein is IGF1(E3R/Y31A)_lk7_HSA25-609(C58S/N527Q)_lk7_AnxV2-320(R63A/K70A/K101A/E138A/D139G/N160A/C316A).

In some embodiments, the linker lk7 comprises or consist of -Gly-Ser-Gly-Gly-Gly-Ser-Gly (SEQ ID NO: 23).

In some embodiments, the method comprises administering daily from about 0.01 mg/kg to about 20 mg/kg of the chimeric protein to the subject in need thereof.

In some embodiments, the method comprises administering a total dose of from about 5 mg/kg to about 20 mg/kg of the chimeric protein to the subject in need thereof over a period of 4 to 7 days.

In some embodiments, the method comprises administering descending doses of the chimeric protein to the subject in need thereof.

In some embodiments, the method comprises administering to the subject in need thereof a first dose comprising from about 2 mg/kg to about 6 mg/kg of the chimeric protein on day 1, and a dose comprising from about 1 mg/kg to about 2 mg/kg one each of the following days.

In some embodiments, the method comprises administering comprises administering to the subject in need thereof a first dose of the pharmaceutical composition on day 1 and a second dose of the pharmaceutical composition on day 2, wherein the first dose comprises an amount of chimeric protein that is higher than an amount of chimeric protein in the second dose, and wherein the first dose comprises from about 2 mg/kg to about 6 mg/kg of the chimeric protein.

In some embodiments, the method comprises administering intravenously, intraarterially, intranigrally, or intrathecally the pharmaceutical composition.

In some embodiments, the method comprises administering intravenously the pharmaceutical composition.

In some embodiments, the method comprises administering intrathecally the pharmaceutical composition.

In some embodiments, the method comprises administering intranigrally the pharmaceutical composition.

Provided herein is a method of treating Parkinson disease, the method comprising administering intrathecally or intranigrally to a subject in need thereof a pharmaceutical composition comprising a therapeutically effective amount of a chimeric protein and a pharmaceutically acceptable carrier, wherein the chimeric protein comprises (a) a targeting domain comprising a variant of human Annexin 5 (AnxV) comprising one or more mutations, wherein the one or more mutations consist of a substitution at the position corresponding to C316 and optionally at one or more positions corresponding to R63, K70, K101, E138, D139, N160, and combinations thereof; (b) an activator domain comprising a variant of human insulin-like growth factor IGF-1 comprising one or more mutations, wherein the one or more mutations consist of a substitution at one or more positions corresponding to E3, Y24, Y31, Y60, and combinations thereof, wherein the variant of IGF-1 decreases activation of the IGF-1 receptor relative to the wild-type IGF-1, and (c) a half-life modulator comprising a variant of human serum albumin (HSA) comprising one or more mutations, wherein the one or more mutations consist of a substitution at one or more positions corresponding to C58 and N527, and combinations thereof.

In some embodiments, the administration of the pharmaceutical composition results in targeted stimulation of the phosphorylation of serine/threonine protein kinase B (AKT) pathway by selective activation of the IGF-1 receptor. In some embodiments, the administration of the pharmaceutical composition results in reduction or prevention of amyloid fibril formation.

In some embodiments, the pharmaceutical composition is free of sucrose. In some embodiments, the pharmaceutical composition is administered intrathecally. In some embodiments, the pharmaceutical composition is administered intranigrally.

In some embodiments, the variant of IGF-1 decreases activation of the IGF-1 receptor relative to the wild-type IGF-1. In some embodiments, the variant of IGF-1 comprises E3R and Y31A substitutions relative to wild type human IGF-1.

In some embodiments, the variant of human Annexin 5 comprises the amino acids 2-320 corresponding to wild type human Annexin 5 and comprises R63A, K70A, K101A, E138A, D139G, N160A, C316A substitutions relative to wild type human Annexin 5.

In some embodiments, the variant of human serum albumin comprises the amino acids 25-609 corresponding to wild type human serum albumin and comprises C58S and N527Q substitutions relative to wild type human serum albumin.

In some embodiments, the chimeric protein is IGF1(E3R/Y31A)_lk7_HSA25-609(C58S/N527Q)_lk7_AnxV2-320(R63A/K70A/K101A/E138A/D139G/N160A/C316A).

In some embodiments, the linker lk7 comprises or consist of -Gly-Ser-Gly-Gly-Gly-Ser-Gly (SEQ ID NO: 23).

In some embodiments, the method comprises administering daily from about 0.01 mg/kg to about 20 mg/kg of the chimeric protein to the subject in need thereof.

In some embodiments, the method comprises administering a total dose of from about 5 mg/kg to about 20 mg/kg of the chimeric protein to the subject in need thereof over a period of 4 to 7 days.

In some embodiments, the method comprises administering descending doses of the chimeric protein to the subject in need thereof.

In some embodiments, the method comprises administering to the subject in need thereof a first dose comprising from about 2 mg/kg to about 6 mg/kg of the chimeric protein on day 1, and a dose comprising from about 1 mg/kg to about 2 mg/kg one each of the following days.

In some embodiments, the method comprises administering comprises administering to the subject in need thereof a first dose of the pharmaceutical composition on day 1 and a second dose of the pharmaceutical composition on day 2, wherein the first dose comprises an amount of chimeric protein that is higher than an amount of chimeric protein in the second dose, and wherein the first dose comprises from about 2 mg/kg to about 6 mg/kg of the chimeric protein.

### DESCRIPTION OF THE SEQUENCE LISTING

SEQ ID NO: 1 is the amino acid sequence of wild-type human IGF-1 (mature form).

SEQ ID NO: 2 is the amino acid sequence of a variant of wild-type human IGF-1 variant comprising E3R and Y31A substitutions.

SEQ ID NO: 3 is the amino acid sequence a variant of human IGF-1 (IGF-1 LONG).

SEQ ID NO: 4 is the amino acid sequence a variant of human IGF-1 (IGF1 E3R).

SEQ ID NO: 5 is the amino acid sequence of a variant of human IGF-1 (IGF-1Des 1-3).

SEQ ID NO: 6 is the amino acid sequence of a variant of human IGF-1 (IGF-1 LR3).

SEQ ID NO: 7 is the amino acid sequence a variant of human IGF-1 (IGF1 R37X).

SEQ ID NO: 8 is the amino acid sequence of human a variant of human IGF-1 with deletion of residues 68-70 (IGF1 3X).

SEQ ID NO: 9 is the amino acid sequence of wild type human annexin A5 (AnxV).

SEQ ID NO: 10 is the amino acid sequence of a variant of wild-type human annexin 5 comprising the amino acids 2-320 of wild type annexin 5 and the R63A, K70A, K101A, E138A, D139G, N160A and C316A substitutions.

SEQ ID NO: 11 is the amino acid sequence of non-internalizing variant of human annexin A5 (ni-AnxV).

SEQ ID NO: 12 is the amino acid sequence of a variant human serum albumin comprising the amino acids 25-609 of wild type human serum albumin (HSA).

SEQ ID NO: 13 is the amino acid sequence of Human Serum Albumin variant mHSA comprising the amino acids 25-609 of wild type Human Serum Albumin and C34S, N503Q substitutions.

SEQ ID NO: 14 is the amino acid sequence of Human Serum Albumin variant mHSA7 comprising the amino acids 25-609 of wild type Human Serum Albumin and C34S, N503Q, E505G and V547A substitutions.

SEQ ID NO: 15 is the amino acid sequence of a variant human serum albumin comprising the amino acids 25-609 of wild type human serum albumin and the C58S and N527Q substitutions.

SEQ ID NO: 16 is the amino acid sequence of a peptide linker.

SEQ ID NO: 17 is the amino acid sequence of human transferrin (Tf).

SEQ ID NO: 18 is the amino acid sequence of Human Alpha Fetoprotein (AFP).

SEQ ID NO: 19 is the amino acid sequence of Human Vitamin D Binding Protein (VDBP).

SEQ ID NO: 20 is the amino acid sequence of Human Transthyretin (TTR).

SEQ ID NO: 21 is the amino acid sequence of a motif PASylation.

SEQ ID NO: 22 is the amino acid sequence of the albumin-binding domain human antibody (aldudAB).

SEQ ID NO: 23 is the amino acid sequence of a peptide linker lk7.

SEQ ID NO: 24 is the amino acid sequence of chimeric protein scp776 IGF1(E3R/Y31A)_lk7_HSA25-609(C58S/N527Q)_lk7_AnxV2-320(R63A/K70A/K101A/E138A/D139G/N160A/C316A.

SEQ ID NO: 25 is the amino acid sequence of chimeric protein IGF1(E3R/Y31A)_lk7_HSA25-609(C58S/N527Q)_lk7_AnxV2-320(R63A/K70A/K101A/E138A/D139G/N160A/C316S).

SEQ ID NO: 26 is the amino acid sequence showing IGF-1 residues identified via application of an algorithm that considers solvent accessibility, hydrophobicity, and temperature facto.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows IGF-1 residues identified via application of an algorithm that considers solvent accessibility, hydrophobicity, and temperature factor. Represented by X are residue position that can be mutated.

FIG. 2 shows hydrophobic core positions of IGF-1.

FIG. 3A shows the neurological deficit score (NDS)-motor system score at 48 hours (day 2) for placebo treated and scp776 treated animals.

FIG. 3B shows the NDS-consciousness score at day 7 for placebo treated and scp776 treated animals.

FIG. 3C shows the NDS-musculoskeletal coordination score at day 1, 2, 3, 7, 10, and 14 for placebo treated (top curve) and scp776 treated (bottom curve) animals.

FIG. 3D shows the total NDS score at day 1, 2, 3, 7, 10, and 14 for placebo treated (top curve) and scp776 treated (bottom curve) animals.

FIG. 4 shows the results of the MRI analysis of the lesion size (mm²) at 8 hours, 72 hours and 14 days for placebo treated and scp776 treated animals.

FIG. 5A shows a rat brain section stained with an anti-human serum albumin antibody 4 hours after direct intranigral injection of 10 μg scp776 (20× magnification).

FIG. 5B shows a rat brain section stained with an anti-human serum albumin antibody 4 hours after direct intranigral injection of 40 μg scp776 (20× magnification).

FIG. 5C shows a rat brain section stained with an anti-human serum albumin antibody 4 hours after direct intranigral injection of 160 μg scp776 (20× magnification).

FIG. 5D shows a portion of the image in FIG. 5C at 600× magnification to highlight the possible uptake of scp776 into select cells (marked with arrows).

### DETAILED DESCRIPTION

It is to be understood that the terminology used herein is for the purpose of describing particular embodiments of the disclosure only and is not intended to be limiting.

Unless defined otherwise, all technical and scientific terms used herein have the same meaning as commonly understood by one having ordinary skill in the art to which the disclosure pertains.

All publications, patents and patent applications cited herein, whether supra or infra, are hereby incorporated by reference in their entirety.

### Definitions

As used in this specification and the appended claims, the singular forms “a, “an” and “the” include plural referents unless the content clearly dictates otherwise.

The term “peptide,” “polypeptide” and “protein” are used interchangeably to denote a sequence polymer of at least two amino acids covalently linked by an amide bond (also referred herein as peptide bond).

As used herein the term “target molecule” refers to any molecule that is associated with a tissue (e.g. “at risk”, diseased or damaged tissue). A “target cell” is meant to be a cell to which a protein or targeting domain thereof can specifically bind.

“Binding” or “specific binding” are used interchangeably herein and indicates that a protein (or the targeting polypeptide domain thereof or the activator domain thereof) exhibits substantial affinity for a specific molecule (e.g., targeting domain exhibits substantial affinity for a target molecule, or an activator domain exhibits substantial affinity for a molecule associated with the surface of a cell such as a growth factor receptor) or a cell or tissue bearing the molecule and is said to occur when the protein (or the targeting polypeptide domain thereof or the activator domain thereof) has a substantial affinity for a specific molecule and is selective in that it does not exhibit significant cross-reactivity with other molecules.

“Identity,” as known in the art, is a relationship between two or more polypeptide or protein sequences, as determined by comparing the sequences. In the art, “identity” also refers to the degree of sequence relatedness between polypeptides or proteins, as determined by the match between strings of such sequences. “Identity” can be readily calculated by any bioinformational methods known in the art.

The term “parent polypeptide” refers to a wild-type polypeptide and the amino acid sequence or nucleotide sequence of the wild-type polypeptide is part of a publicly accessible protein database (e.g., EMBL Nucleotide Sequence Database, NCBI Entrez, ExPasy, Protein Data Bank and the like).

The term “mutant polypeptide” or “polypeptide variant” refers to a form of a polypeptide, wherein its amino acid sequence differs from the amino acid sequence of its corresponding wild-type (parent) form, naturally existing form or any other parent form. A mutant polypeptide can contain one or more mutations, e.g., substitution, insertion, deletion, addition, etc., which result in the mutant polypeptide. Generally, variants are overall closely similar, and, in many regions, identical to the reference polypeptide. As used herein, “variant” refers to a polypeptide, differing in sequence from a native protein but retaining at least one functional and/or therapeutic property thereof as described elsewhere herein or otherwise known in the art.

The term “corresponding to a parent polypeptide” is used to describe a polypeptide of the disclosure, wherein the amino acid sequence of the polypeptide differs from the amino acid sequence of the corresponding parent polypeptide only by the presence of at least one amino acid variation. Typically, the amino acid sequences of the variant polypeptide and the parent polypeptide exhibit a high percentage of identity. In one example, “corresponding to a parent polypeptide” means that the amino acid sequence of the variant polypeptide has at least about 50% identity, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98% identity or at least about 99% identity to the amino acid sequence of the parent polypeptide. In another example, the nucleic acid sequence that encodes the variant polypeptide has at least about 50% identity, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98% identity or at least about 99% identity to the nucleic acid sequence encoding the parent polypeptide.

The term “substantial identity” or “substantial similarity,” as used herein, when referring to a nucleic acid or fragment thereof, indicates that when optimally aligned with appropriate nucleotide insertions or deletions with another nucleic acid (or its complementary strand), there is nucleotide sequence identity in at least about 95% to 99% of the sequence. The term “substantial identity” or “substantial similarity,” as used herein, when referring to a protein or fragment thereof, indicates that when optimally aligned there is an amino acid sequence identity in at least about 95% to 99% of the sequence.

The term “damaged cell” or “damaged tissue,” as used herein, means and includes biological cell or tissue; for example, but not limited to, neurons, glia or nervous tissue damaged or injured by, but not limited to, trauma or chemical insult, ischemic tissue, cell or tissue damaged by any means which results in interruption of normal blood flow to the tissue.

The term “therapeutically effective amount,” as used herein, means the amount of the protein or agent that will elicit the biological or medical response of a tissue, system, animal or human that is being sought by the researcher, veterinarian, medical doctor or other clinician.

The term “pharmaceutically acceptable,” or “physiologically acceptable” as used herein, means the carrier, diluent or excipient must be compatible with the other ingredients of the formulation and not deleterious to the recipient thereof.

The term “targeting moiety”, “targeting domain,” “targeting polypeptide” or “targeting module” are used herein interchangeably and refer to molecules that selectively localize the chimeric protein in a particular tissue or region of the body. The localization can be mediated by specific recognition of molecular determinants, molecular size of the targeting domain, ionic interactions, hydrophobic interactions and the like. As used herein, the terms “therapeutic moiety,” “activator domain,” “activator polypeptide”, “signaling arm” and “effector module” are used herein interchangeably and refers to any agents useful for therapy and that are non-toxic, do not have a cytotoxic effect or are not detrimental to the cells. Such agents can include, but not limited to, growth factors.

### Chimeric Proteins

Chimeric proteins provided herein are capable of specific binding to two or more different specific molecules. In some embodiments, the chimeric protein comprises a targeting domain having a binding specificity to a first specific target molecule, an activator domain having a binding specificity to a second target molecule, and a half-life modulator.

In some aspects, the activator domain has a binding specificity to a tyrosine kinase receptor at the cell surface. In some embodiments, the binding of the activator to the tyrosine kinase receptor activates intracellular signaling pathways associated with cell survival. In some aspects, the activator domain has a binding specificity to a receptor that modulates/promotes tissue regeneration.

In some embodiments, the targeting domain serves to target the chimeric protein to a target cell or tissue while the activator domain serves to activate the intracellular signaling pathway associated with cell survival.

In some embodiments, the half-life modulator extends the half-life of the chimeric protein.

In some embodiments, the chimeric proteins are fusion proteins having a targeting polypeptide connected or linked to a half-life modulator and to an activator polypeptide. In some embodiments, the engineered proteins are chimeric proteins having a targeting polypeptide connected or linked to a half-life modulator and a growth factor or mutated growth factor.

In some embodiments, the mutated growth factor (e.g. IGF-1 variant) is engineered to reduce potency while retaining the ability to activate the cognate growth factor receptor. In some embodiments, wild type growth factors can be used as activator domains.

The targeting domain is generally used to target the chimeric proteins to a target cell. In some embodiments the target cell is undergoing apoptosis. The binding of the targeting domain to its target molecule does not induce a significant biological effect in the target cell. The activator domain binds to a receptor on a cell surface. The binding of the activator domain to its receptor is intended to modulate a specific biological effect, such as, activate the intracellular signaling pathway associated with cell survival. In some embodiments, binding of the activator domain to its receptor is intended the positively regulate survival of the targeted cells or tissue. In particular, the activator domain of chimeric protein can promote survival signaling.

In some embodiments, the in vivo activity of the chimeric protein can be assessed by detecting signaling changes in molecules that are regulated by the activator domain, including but not limited to cell surface receptor phosphorylation status or downstream mediators such as phospho-AKT or phospho-ERK (as detected by flow cytometry, immunofluorescence, ELISA, phospho-labeling, Western analysis of treated tissues, or any other methodology known in the art.) In some embodiments, a chimeric protein functions in vivo if it induces a significant (e.g., at least 10%, at least 20%, at least 30%, at least 40%, at least 50% or more) change in the level, functional activity, or phosphorylation of the regulated molecule detected by the assay.

### Activator Domain

The activator domain can be any polypeptide that detectably modulates the activity of a cellular network. In some embodiments, the activator domain is capable of activating signal transduction pathways by binding to a receptor at the surface a cell. In some embodiments, certain activator domains are growth factor polypeptides, or any agonist of the receptor. It will be apparent that such modulation may be an increase in the activity of the cellular network such as induction of proliferation of cells, induction of cell growth, promotion of cell survival and/or inhibition of apoptosis.

An activator domain for a particular application may be selected based on the desired therapeutic outcome. For example, to increase survival and neuroprotection, activator domains that comprise IGF-1 (or variant or fragment thereof), NRG1 (or variant or fragment thereof), FGF2 (or variant or fragment thereof), FGF9 (or variant or fragment thereof), CCL4 (or variant or fragment thereof), Hepatocyte Growth Factor (NK1 or fragment thereof), Vascular Endothelial Growth Factor A (or variant or fragment thereof), BMP2 (or variant or fragment thereof), can be used.

In some embodiments, the activator domain comprises a change in the amino acid sequence, the three-dimensional structure of the protein, and/or the activity of the protein, relative to the wild-type form of the protein.

In some embodiments, the activator domain comprises or consists of a growth factor having amino acid sequence modification relative to the wild-type growth factor (e.g. IGF-1) to decrease its binding to its natural receptor (e.g. IGF-1 receptor), to decrease its binding to binding proteins (e.g. IGF binding proteins) and/or decrease its activation of its natural receptor (e.g. IGF-1 receptor). In some embodiments, the activator domain is a growth factor having amino acid sequence modification that reduce (e.g., for about 1-5%, 5-10%, 10%-20%, about 20%-40%, about 50%, about 40%-60%, about 60%-80%, about 80%-90%, 90-95%) the binding to its natural receptor (e.g. IGF-1 receptor).

A growth factor polypeptide detectably modulates activation of a growth factor receptor. In some embodiments, the activator domain of the bi-specific protein is a growth factor, variant or fragment thereof that retains at least about 0.01% of wild-type biological activity. In some embodiments, the activator domain of the bi-specific protein is a growth factor, variant or fragment thereof that retain at least about 0.1%, at least about 1%, at least about 10%, of wild-type biological activity. In some embodiments, the activator domain of the bi-specific protein is a growth factor, variant or fragment thereof that retains between about 0.01% to about 0.1% of wild-type biological activity. In some embodiments, the activator domain of the bi-specific protein is a growth factor, variant or fragment thereof that retains between about 0.01% to about 1% of wild-type biological activity. In some embodiments, the activator domain of the bi-specific protein is a growth factor, variant or fragment thereof that retains between about 0.01% to about 10% of wild-type biological activity. In some embodiments, the activator domain of the bi-specific protein is a growth factor, variant or fragment thereof that retains between about 0.1% to about 1% of wild-type biological activity. In some embodiments, the activator domain of the bi-specific protein is a growth factor, variant or fragment thereof that retains between about 0.1% to about 10% of wild-type biological activity. In some embodiments, the activator domain of the bi-specific protein is a growth factor, variant or fragment thereof that retains between about 01% to about 10% of wild-type biological activity. Biological activity in some embodiments can be determined by measuring activation of the corresponding growth factor receptor in appropriate cells. In some embodiments, activation may be assessed, for example, by measuring phosphorylation of receptor kinase or downstream effector proteins, such as, but not limited to, AKT, S6, ERK, JNK, mTOR, etc.

### Insulin-Like Growth Factors (IGFs) and Derivatives Thereof

The insulin-like growth factors (IGFs) constitute a family of proteins having insulin-like and growth stimulating properties. The IGFs Human IGF-1 is a 70 amino acids basic peptide having the protein shown in SEQ ID NO: 1, respectively. IGF-1 and extracellular tyrosine kinase receptor (e.g. IGF-1 receptor) are important for cellular processes such as cell proliferation and survival. Binding of IGF-1 or variant thereof to the IGF-1 receptor stimulates kinase activity, leading to phosphorylation of multiple substrate, thereby initiating signaling cascades. The chimeric proteins disclosed herein can maintain the ability to signal through the extracellular receptor, for example IGF-1 receptor. The activator domain IGF-1 stimulates cell proliferation and survival through activation of the AKT pathway. Upon binding of IGF-I to the IGF-1 receptor, a tyrosine kinase, phosphorylates tyrosine residues on two major substrates, IRS-1 and Shc, which subsequently signal through the Ras/Raf and PI 3-kinase/AKT pathways.

The interaction of IGF-1 (and IGF-2) with the IGF-1 receptor is regulated by IGF binding Proteins (IGFBPs). All six IGFBPs (particularly IGFBP5) have been shown to inhibit IGF action, but in some instances a stimulatory effect has been observed. At least 99% of the IGF in the circulation is normally bound to IGFBPs.

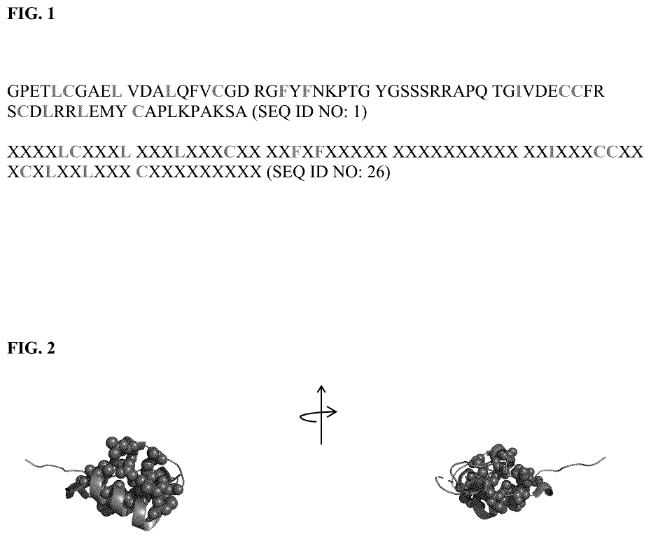

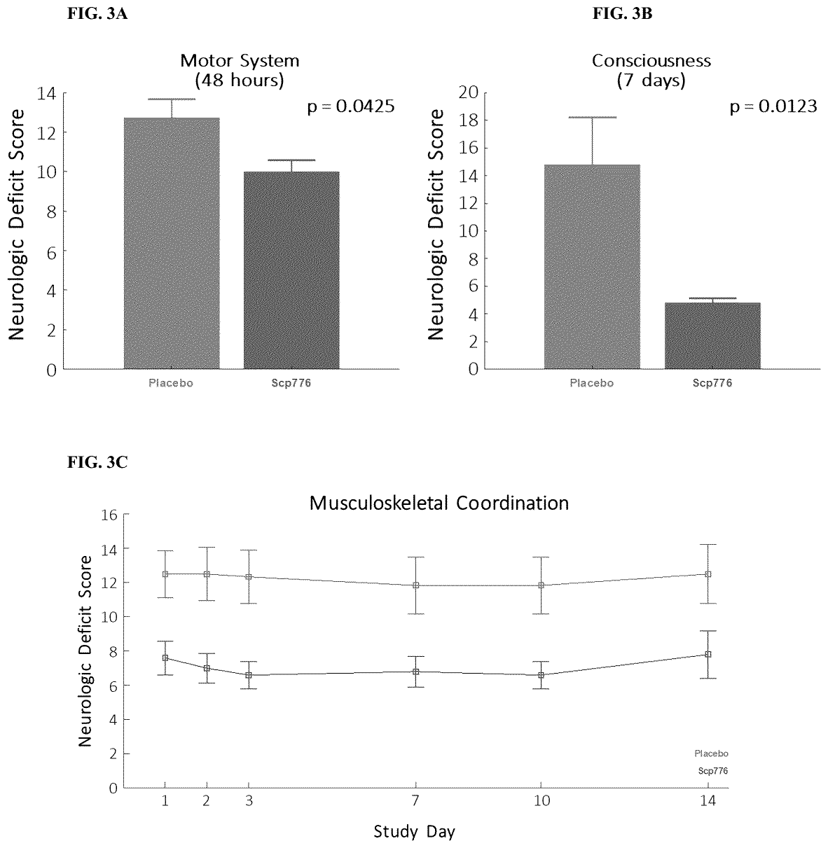

In some embodiments, the activator domain is a variant of the human IGF-1 or fragment thereof. In some embodiments, the variant of IGF-1 or fragment thereof is capable of maintaining selectivity to the IGF-1 receptor. FIG. 1 shows residues identified via application of an algorithm that considers solvent accessibility, hydrophobicity, and temperature factor. Represented by X are residue position that can be mutated. Collectively, these residues represent the hydrophobic core of IGF-1. Hydrophobic core positions of IGF-1 are depicted as spheres (pdb model 1H02) at FIG. 2.

In some embodiments, the IGF-1 variant is modified to reduce binding to IGF-1 binding proteins (IGFBPs) relative to wild-type IGF-1 while maintaining its ability to activate the AKT pathway. In some embodiments, the IGF-1 variant can activate the IGF-1 receptor with a decreased potency for non-target cells, as assessed by pAKT EC50. EC50 is defined as the concentration needed to achieve the half maximal level of pAKT signaling.

In some embodiments, the IGF-1 variant comprises a substitution at one or more of the tyrosine residues. In some embodiments, the IGF-1 variant comprises one or more substitutions at position Y24, Y31 and Y60. In an exemplary embodiment, the IGF-1 variant can comprise a single tyrosine substitution at position Y31, or Y24, or Y60. In an exemplary embodiment, the IGF-1 variant can comprise a single tyrosine substitution at position Y24 and Y31, Y24 and Y60, Y31 and Y60, or Y24 and Y60. In another exemplary embodiment, the IGF-1 variant can comprise one or more of the following substitutions, Y24L, Y31A, and Y60L relative to wild type IGF-1. For example, the IGF-1 variant can comprise the Y24L substitution and the Y31A substitution or the IGF-1 variant can comprise the Y24L substitution, the Y31A substitution and the Y60L. In some embodiments, one or more tyrosine residues (Y24, Y31, Y60 or combinations thereof) can be substituted for a short aliphatic amino acid. In some embodiments, one or more tyrosine residues (Y24, Y31, Y60 or combinations thereof) can be substituted for a polar amino acid. In some embodiments, one or more tyrosine residues (Y24, Y31, Y60 or combinations thereof) can be substituted for leucine, alanine, isoleucine, serine, threonine or any other amino acid.

In some embodiments, the IGF-1 variant comprises a substitution replacing Arg for a Glu, Lys, Met, Val, Ala, Leu, Ile, Gly, Ser, or Thr at the 3 position of the polypeptide. In some embodiments, the IGF-1 variant comprises a substitution replacing Arg for a Glu at the 3 position of the polypeptide (E3R).

In some embodiments, the IGF-1 variant comprises a substitution at the position 3 and 31. For example, the IGF-1 variant comprises E3R and Y31A substitutions. In some embodiments, the activator domain has an amino acid sequence having SEQ ID NO: 2.

In some embodiments, the activator domain is a derivative of the human IGF-1 comprising one or more of the following modifications: a N-terminal 13-residue extension (IGF-1 LONG), a deletion of amino acids 1-3 (Des-1-3), a substitution replacing Arg for a Glu at the 3 position of the polypeptide (E3R), no Arginine at position 37 (R37X), a deletion of amino acids 68-70 (3X), an N-terminal 13-residue extension and a substitution replacing Arg for a Glu at the 3 position of the wild-type polypeptide (LR3), substitutions of one or more of tyrosine residues (Y24, Y31, Y60 or combinations thereof (e.g. Y24L, Y31A, Y60L substitutions or combinations thereof).

In some embodiments, the activator domain is variant of the human IGF-1 comprising a mutation (e.g. substitution, deletion) at one or more residues 24 to 37.

In some embodiments, the activator domain is a derivative of the human IGF-1 and comprises an N-terminal 13-residue extension (also referred as IGF-1 LONG, SEQ ID NO: 3), a mutation E3R (SEQ ID NO: 4) or a combination thereof (LONG E3R, also referred as LR3, SEQ ID NO: 6). In some embodiments, the IGF-1 variant comprises the E3R substitution, an N-terminal 13-residue extension, deletion of amino acids 1-3 ((Des1-3), SEQ ID NO: 5) or a combination thereof to decrease the binding of the activator domain to the IGF binding proteins which are present in the serum and other body fluid.

In some embodiments, the activator domain is a derivative of the human IGF-1 and comprises one or more of the following modifications: an N-terminal 13-residue extension (SEQ ID NO: 3), a deletion of amino acids 1-3 (SEQ ID NO: 5), a substitution replacing Arg for a Glu at the 3 position of the polypeptide (SEQ ID NO: 4), no Arginine at position 37 (R37X, SEQ ID NO: 7), a deletion of amino acids 68-70 (3X, SEQ ID NO: 8), or an N-terminal 13-residue extension and a substitution replacing Arg for a Glu at the 3 position of the wild-type polypeptide (SEQ ID NO: 6).

It is believed that the bi-specific proteins that contain the variant of IGF-1 described herein (e.g. E3R, IGF-1 LONG, IGF-1 LONG E3R (referred to as IGF-1(LR3)) or IGF1 Des1-3), have decreased affinity for IGF binding proteins relative to wild-type IGF-1. In some embodiments, the IGF-1 variants of the bi-specific proteins described herein can activate the signaling pathway while having a substantially decreased interaction with the IGF-1 binding proteins relative to wild-type IGF-1.

In some embodiments, the IGF-1 variant can be modified by glycosylation of one or more glycosylation site present in the IGF-1 variant.

In some embodiments, the chimeric proteins that contain the IGF-1 variants described herein have a potency for non-target cells that is less than wild-type IGF-1 for non-target cells.

Certain activator domains that bind to growth factor receptors are provided herein in SEQ ID NOs: 1-8.

Additional peptide sequence modifications can be included, such as variations, deletions, substitutions or derivatizations of the amino acid sequence of the sequences disclosed herein, so long as the peptide has substantially the same activity or function as the unmodified peptides. Notably, a modified peptide will retain activity or function associated with the unmodified peptide, the modified peptide will generally have an amino acid sequence “substantially homologous” with the amino acid sequence of the unmodified sequence.

In some embodiments, the IGF-1 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NOs: 1-8. In some embodiments, the IGF-1 variant can have an amino acid sequence having from about 85% to about 90%, from about 90% to about 95%, from about 95% to about 98%, from about 98% identity to about 99% identity to the amino acid sequence provided in SEQ ID NOs: 1-8. In some embodiments, the IGF-1 variant can comprise 10, 20, 30, 40, 50, 60 or more consecutive amino acids of any one of amino acids in SEQ ID NOs: 1-8. In some embodiments, the IGF-1 variant can have an amino acid sequence recited in any one of SEQ ID NOs: 1-8. In some embodiments, the IGF-1 variant can have an amino acid sequence recited in any one of SEQ ID NOs: 2-8 In some embodiments, the IGF-1 variant can have an amino acid sequence recited in SEQ ID NO: 2.

In some embodiments, the bi-specific protein comprises an activator domain having a growth factor variant that is selected to give the bi-specific protein at least an order of magnitude lower EC50 in damaged tissue than in healthy tissue. For example, the bi-specific protein domain comprises a growth factor variant and has an EC50 in damaged tissue that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower than the EC50 in healthy tissue.

In some embodiments, the bi-specific proteins that contain the IGF-1 variants have a half maximal effective concentration (EC50) that is lower in damaged tissue than in healthy tissue. In some embodiments, the bi-specific proteins that contain the IGF-1 variants have a half maximal effective concentration (EC50) that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower in damaged tissue than in healthy tissue.

In some embodiments, the chimeric proteins provided herein having such variant growth factors have a higher specificity to the damaged tissue targeted.

### Target Molecules

In some embodiments, target molecules are exposed or enriched on the exterior of a target cell. In some embodiments, the target molecule is associated with a damaged cell, early apoptotic or apoptotic cell, the target molecule being intracellular in a viable or undamaged cell and being exposed to the extracellular space in a damaged cell. Such molecules include, for example, molecules that are exposed in cells that undergo necrosis (such as DNA) or apoptosis (e.g., phosphatidylserine), myosin (including the tissue type-specific subtypes thereof), ICAM-1 or P-selectin. Yet in other embodiments, the target molecule is a molecule that is present or enriched at the surface of a diseased or dysfunctional cell or tissue as compared to the level detected in a healthy or functional cell or tissue. In some embodiments, the target cell is not a tumor or cancerous cell.

Cells are bounded by a plasma membrane (or cell membrane) comprising a lipid bilayer. The cell membrane may be considered to have a surface facing the cytosol (cytosolic side or interior of the cell) and a surface facing the exterior of the cell, or the extracellular space. Trans-bilayer movement of anionic phospholipids from the inner to the outer leaflet of the plasma membrane occurs during apoptosis. The anionic phospholipid-binding protein, such as Annexin A5, synaptotagmin I or lactadherin can be used to detect the presence of phosphatidylserine on the outer leaflet of the cell membrane. Phosphatidylserine is a phospholipid, that is usually restricted to the cytosolic side of the membrane in viable or undamaged cells, and that becomes exposed on the outer cell surface or to the extracellular space in damaged cells or apoptosis.

In some embodiments, the target molecule is an “ischemia-associated molecule”. An “ischemia-associated molecule” is any molecule that is detected at a level that is significantly higher (e.g., at least 1.5 higher, at least 2-fold higher, at least 3-fold higher, at least 4-fold higher, at least 5-fold higher) following ischemia (which results in hypoxia) or hypoxia than in a cell of the same tissue that has not undergone an ischemic event (i.e., the molecule is specific to or enriched in the post-ischemic tissue). Ischemia occurs when there is insufficient blood flow to provide adequate oxygenation, which results in tissue hypoxia (reduced oxygen) or anoxia (absence of oxygen) as the most severe form of hypoxia, and ultimately tissue necrosis, and apoptosis.

### Targeting Domain

In some embodiments, the targeting domain has a specific binding affinity to a target molecule associated with a tissue (for example, an ischemia-associated molecule). In some embodiments, the targeting domain has a specific binding affinity for a target molecule presented on the surface of early apoptotic cells. The targeting domain may be any polypeptide sequence that serves this function. In some embodiments, binding of the targeting domain to the target molecule does not have or does not modulate a biological activity. As used herein, “biological activity” refers to a defined, known activity performed by exposure of a molecule to a domain of the protein.

In some embodiments, the targeting domain can be a non-antibody polypeptide, fragment thereof or variant thereof having a binding affinity to the target molecule. Yet in other embodiments, the targeting polypeptide domain comprises one or more antibody variable regions (e.g. scFv).

### Annexin A5 and Variants Thereof

In some embodiments, the targeting domain comprises annexin, a variant thereof or a fragment thereof. The term “annexin” refers to any protein capable of binding to phospholipids, especially phosphatidylserine (PS), and member of the annexin family. In some embodiments, the annexin is Annexin A5 but other annexins can equally be used. In some embodiments, the targeting domain is human Annexin A5, a functional fragment thereof, or a variant thereof. A variant of Annexin A5 comprises at least one amino acid in at least one position in which this amino acid is not found in the parent wild type Annexin A5 polypeptide (SEQ ID NO: 9). The annexin variants according may comprise one or more amino acid substitutions, deletions, additions, or combinations thereof wherein the amino acid substitutions, deletions, or additions do not substantially affect the ability of the Annexin A5 variant of the chimeric protein to bind to at least one phospholipid, such as PS. In some embodiments, the Annexin A5 variant can have an amino acid sequence having at least about 85%, at least about 90%, at least about 95%, at least about 98% identity or at least about 99% identity to the amino acid sequence provided in SEQ ID NO: 9. In some embodiments, the Annexin A5 variant can comprise 50, 80, 100, 110, 200, 300, or more consecutive amino acid having at least about 85%, at least about 90%, at least about 95%, at least about 98% identity or at least about 99% identity to the amino acids in SEQ ID NO: 9.

In some embodiments, the variant of Annexin A5 is modified to substitute cysteine at position 315 (corresponding to C316) with serine or alanine to reduce dimer formation. For example, the cysteine can be substituted to an alanine or a serine. As used herein, the term “corresponding to” is used to designate the position/identity of an amino acid residue in a polypeptide (e.g., Annexin A5). Those of ordinary skill will appreciate that, for purposes of simplicity, a canonical numbering system (based on wild-type Annexin A5) is utilized herein, so that an amino acid “corresponding to” a residue at position 316, for example, need not actually be the 316th amino acid in a particular amino acid chain but rather corresponds to the residue found at position 316 in a for example Annexin A5 before the post-translational removal of the N-terminal methionine; those of ordinary skill in the art readily appreciate how to identify corresponding amino acids. In particular, it is noted that the amino acid sequence of wild-type Annexin A5 (SEQ ID NO: 9) do not start with a Methionine as the Methionine residue is cleaved during processing.

In some embodiments, the variant of Annexin A5 has an amino acid sequences that has been mutated to reduce internalization of Annexin A5 or the chimeric protein comprising the variant of Annexin A5 into a cell while maintaining binding affinity to phosphatidylserine (PS). In some embodiments, the variant of Annexin A5 or the chimeric protein comprising the variant of Annexin A5 has a binding affinity to phosphatidylserine, and is not internalized into a cell or is internalized at a slower rate than wild-type annexin A5. In some embodiments, the targeting domain is a non-internalizing variant of Annexin A5, (also referred as ni-Annexin A5 or ni-Anx V, SEQ ID NO: 11). In some embodiments, the variant of Annexin A5 has an amino acid set forth in SEQ ID NO: 10. In some embodiments, the non-internalizing mutant of Annexin A5 has an amino acid sequence having at least about 85%, at least about 90%, at least about 95%, at least about 98% identity or at least about 99% identity to the amino acid sequence provided in SEQ ID NO: 11. In some embodiments, the non-internalizing mutant of Annexin A5 can have an amino acid sequence having from about 85% to about 90%, from about 90% to about 95%, from about 95% to about 98%, from about 98% to about 99% identity to the amino acid sequence provided in SEQ ID NO: 11. In some embodiments, the Annexin A5 variant can comprise 50, 80, 100, 110, 200, 300, or more consecutive amino acid of any one of amino acids in SEQ ID NO: 11. Any variation of Annexin A5 that results in substantially no internalization is envisioned.

It should be appreciated that the non-internalizing variant of annexin A5 can confer an extended half-life to the chimeric protein as compared to a chimeric protein that contains wild-type A5. In some embodiments, the variants of annexin A5 that results in substantially no internalization, or chimeric proteins containing variants of annexin A5 that results in substantially no internalization, can have an extended half-life of 1.1 to 1.2, 1.1 to 1.3, 1.1. to 1.4, 1.1 to 1.5, 1.1 to 1.6, 1.1 to 1.7, 1.1 to 1.8, 1.1 to 1.9, 1.1 to 2 or greater as compared to wild-type annexin A5, or chimeric proteins containing wild-type annexin A5.

The terms “non-internalizing” and “substantially no internalization,” as used herein, refer to a lack of internalization of a substantial amount of the chimeric protein disclosed herein. For example, the phrase “substantially no internalization” will be understood as less than 50% of the chimeric protein being internalized by a cell to which the chimeric protein is bound, or less than 25% of the chimeric protein being internalized by a cell to which the chimeric protein is bound, or less than 10% of the chimeric protein being internalized by a cell to which the chimeric protein is bound, or less than 5% of the chimeric protein being internalized by a cell to which the chimeric protein is bound, or less than 3% of the chimeric protein being internalized by a cell to which the chimeric protein is bound, or less than 1% of the chimeric protein being internalized by a cell to which the bi-specific protein is bound.

In some embodiments, the non-internalizing mutant of Annexin A5 can have an amino acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% identity or at least about 99% identity to human Annexin A5. In some embodiments, the non-internalizing variant of Annexin A5 comprises a substitution at position 315 (corresponding to C316) wherein the cysteine residue is substituted with serine (Ser), alanine (Ala), leucine (Leu), phenylalanine (Phe), methionine (Met) or tryptophan (Trp). In some embodiments, the non-internalizing mutant of Annexin A5 can have an amino acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% identity or at least about 99% identity to human Annexin A5. In some embodiments, the non-internalizing variant of Annexin A5 comprises a substitution at position 315 (corresponding to C316) wherein the cysteine residue is substituted with serine (Ser) or alanine (Ala). In some embodiments, the non-internalizing mutant of Annexin A5 can have an amino acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% identity or at least about 99% identity to human Annexin A5 modified to substitute cysteine at position 315 (corresponding to C316) with serine or alanine.

In some embodiments, Annexin A5 or Annexin A5 variants (for example variant having a substitution at position C316) are modified to comprise one or more substitutions at the following positions: R62, K69, K100, E137, D138, N159, L313 (corresponding to R63, K70, K101, E138, D139, N160, L314 relative to wild type human Annexin A5).

In some embodiments, the Annexin A5 variant comprises:
R62A, R62E, R62D, R62M, R62L, R62I, R62Y (corresponding to R63A, R63E, R63D, R63M, R63L, R63I, R63Y relative to wild type human Annexin A5);
K69A, K69E, K69D, K69M, K69L, K69I, K69Y (corresponding to K70A, K70E, K70D, K70M, K70L, K70I, K70Y relative to wild type human Annexin A5);
E137A, E137K, E137R, E137M, E137L, E137I, E137Y (E138A, E138K, E138R, E138M, E138L, E138I, E138Y relative to wild type human Annexin A5);
D138G, D138K, D138R, D138M, D138L, D138I, D138Y (corresponding to D139G, D139K, D139R, D139M, D139L, D139I, D139Y relative to wild type human Annexin A5)
N159A, N160M, N160L, N160I, N160V, N160Y (corresponding to N160A, N160M, N160L, N160I, N160V, N160Y relative to wild type human Annexin A5)
L313E L313D, L313K, L313R, L313H, L313Q, L313N, L313Y (corresponding to L314E L314D, L314K, L314R, L314H, L314Q, L314N, L314Y relative to wild type human Annexin A5);
or any combinations of the foregoing.

In some embodiments, Annexin A5 or Annexin A5 variants comprise one or more substitutions at position D143 and/or E227. In some embodiments, Annexin A5 variant comprises:
D142G, D142A, D142K, or D142R (corresponding to D143G, D143A, D143K, or D143R) substitution, and/or
E226G, E226A, E226K, or E226R (corresponding to E227G, E227A, E227K, E227R) substitution.

In some embodiments, Annexin A5 or Annexin A5 variants (for example having a substitution at C316, D143 and/or E227) are modified to comprise one or more of the following substitutions R62A, K69A, K100A, E137A, D138G, N159A, L313E (corresponding to R63A, K70A, K101A, E138A, D139G, N160A, L314E). For example, Annexin A5 having SEQ ID NO: 9 can be modified to have C315A or C315S substitution (corresponding to C316A or C316S relative to wild type Annexin A5) and one or more of the following substitutions R62A, K69A, K100A, E137A, D138G, N159A, L313E (corresponding to R63A, K70A, K101A, E138A, D139G, N160A, L314E relative to wild type Annexin A5).

In some embodiments, human Annexin A5 (SEQ ID NO: 9) are modified to comprise one or more of the following substitutions R62A, K69A, K100A, E137A, D138G, N159A, D143N, E227A, C315S or C315A (corresponding to R63A, K70A, K101A, E138A, D139G, D144N, N160A, E228A, C316S or C316A relative to wild type Annexin A5).

In some embodiments, the targeting domain is Annexin A5 which has been engineered to have R63A, K70A, K101A, E138A, D139G, N160A and C316A or C316S substitutions relative to wild type Annexin A5. For example, the targeting domain can have the amino acid sequence of SEQ ID NO: 10.

In some embodiments, the Annexin A5 variant comprises one, two, three, four, five, six, seven, eight, nine, ten, eleven, twelve, thirteen, fourteen, fifteen, sixteen, seventeen, eighteen nineteen, twenty or more substitutions in different regions, in order to further decrease the internalization of the annexin in a cell. For example, the Annexin A5 variants may comprise R62A and K69A, R62A and K100A, R62A and E137A, R62A and D138G, R62A and N159A, R62A and K69A and K100A, R62A and K69A and E137A, R62A, K69A and K100A, R62A, K69A, K100A, and E137A etc. . . . .

The annexin variants according may further comprise one or more amino acid substitutions, deletions, or additions, wherein the amino acid substitutions, deletions, or additions do not substantially affect the ability of the Annexin A5 variant of the chimeric protein to bind to at least one phospholipid, such as PS.

Native polypeptide can be used as targeting domains. It will be apparent, however, that portions of such native sequences and polypeptides having altered sequences may also be used, provided that such polypeptides retain the ability to bind the target molecule with an appropriate binding affinity (Kd) as described in more details below.

### Antibody Targeting Domain:

In some embodiments, an anti-phosphatidylserine antibody can be used as a targeting domain. As used herein, term “antibody” includes but is not limited to: (i) a Fab fragment, a monovalent fragment consisting of the VL, VH, CL and CH1 domains; (ii) F(ab)2 and F(ab′)2 fragments, a bivalent fragment comprising two Fab fragments linked by a disulfide bridge at the hinge region; (iii) a Fd fragment consisting of the VH and CH1 domains; (iv) a scFv fragment consisting of the VL and VH domains of a single arm of an antibody, (v) a dAb fragment which consists of a VH domain; and (vi) an isolated complementarity determining region (CDR). Such antibodies may be produced from intact antibodies using methods known in the art, or may be produced recombinantly, using standard recombinant DNA and protein expression technologies.

### Binding of Targeting Domain

In some embodiments, the chimeric protein binds to the target molecule with a K_{d} of less than 10⁻⁶ M, preferably less than 10⁻⁷ M, 10⁻⁸ M, 10⁻⁹ M or 10⁻¹⁰ M.

### Half-Life Modulator

One skilled in the art would appreciate that proteins used in therapeutic applications may not exhibit optimal serum half-lives due to their relatively low molecular weight. In some therapeutic applications, it may therefore be desirable to extend the half-life of the proteins. In some embodiments, to achieve accumulation of the chimeric protein to the diseased injured or damaged area of an organ, the chimeric protein is conjugated operatively associated or fused with a half-life modulator. Preferably, the half-life modulator is non-immunogenic polypeptide.

For example, short half-life is the most limiting attribute of wild-type growth factors as therapeutics. Intravenous administered IGF-1 has a serum half-life in humans of less than 1 hour. The extended half-life of chimeric proteins disclosed herein compared to IGF-1, for example, allows for 1) equivalent efficacy with less frequent dosing; 2) equivalent exposure at a lower dose; 3) lower Cmax at an equivalent exposure level, reducing the risk of Cmax-related toxicity.

In some embodiments, the half-life modulators can increase the in vivo half-life of the chimeric proteins. For example, the half-life of the chimeric proteins comprising the half-life modulator is about 1 hour, 2 hour, 3 hours, 4 hours, 5 hours, 6 hours or greater. For example, the half-life of the chimeric proteins can be about 8 hours or more when tested in Cynomolgus monkey. In some embodiments, the half-life of the chimeric proteins comprising the half-life modulator is about 24 hours, or greater. In some embodiments, the half-life of the chimeric proteins comprising the half-life modulator is about a week or greater.

In some embodiments, the half-life modulator is non-immunogenic in humans.

In some embodiments, the half-life modulator is a polypeptide that interacts with cellular machinery that promote evasion of lysosomal degradation pathways (e.g.—FcRn receptor-mediated recycling).

In some embodiments, the half-life modulator is designed to extend the half-life of the chimeric protein through binding to serum components such as Human Serum Albumin (HSA). HSA is the most abundant protein in the blood and has a demonstrated safety in humans.

In some embodiments, the half-life modulator is an HSA variant. In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild type human serum albumin amino acid sequence (wtHSA, SEQ ID NO: 12). In some embodiments, the half-life modulator comprises at least 200 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 300 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 400 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 500 consecutive amino acids that are at least 70%, 80%, 85%, 90%, 91%, 92%, 93%, 94%, 95%, 96%, 97%, 98%, or 99% identical to wild type human serum albumin amino acid sequence.

In some embodiments, the HSA variant can have one of more of the following substitutions:
cysteine C58 can be substituted, for example, with serine (C58S), alanine (C58A), asparagine (C58N), leucine (C58L), or glutamine (C58Q),
lysine K420 can be substituted for example, with glutamic acid (K420E), aspartic acid (K420D), a leucine (K420L) or methionine (K410M),
asparagine N527 can be substituted for example, with glutamine (N527Q), aspartic acid (N527D), histidine (N527H), or tyrosine (N527Y),
glutamic acid E505 can be substituted for example, with glycine (E505G), alanine (E505A), leucine (E505L), lysine (E505K), valine (E505V), isoleucine (E5051), methionine (E505M), or glutamine (E505Q),
valine V547 can be substituted for example, with alanine (V547A), glycine (V547G), leucine (V547L), lysine (V547K), isoleucine (V547I), methionine (V547M),
asparagine N503 can be substituted for example, with a Glutamine (N527Q), aspartic acid (N503D), histidine (N503H), or tyrosine (N503Y), or glutamine (V547Q).

In some embodiments, the HSA variant can have amino acids 25-609 and have one of more of the following substitutions:
cysteine C58 can be substituted for example, with serine (C58S), alanine (C58A), asparagine (C58N), leucine (C58L), or glutamine (C58Q),
lysine K420 can be substituted for example, with glutamic acid (K420E), aspartic acid (K420D), a leucine (K420L) or methionine (K410M),
asparagine N527 can be substituted for example, with glutamine (N527Q), aspartic acid (N527D), histidine (N527H), or tyrosine (N527Y),
glutamic acid E505 can be substituted for example, with a glycine G (E505G), alanine (E505A), leucine (E505L), lysine (E505K), valine (E505V), isoleucine (E505I), methionine (E505M), or glutamine (E505Q),
valine V547 can be substituted for example, with an alanine (V547A), glycine (V547G), leucine (V547L), lysine (V547K), isoleucine (V547I), methionine (V547M),
asparagine N503 and/or N527 can be substituted for example, with Glutamine (N503Q and/or N527Q), aspartic acid (N503D and/or N527D), histidine (N503H and/or N527H), or tyrosin (N503Y and/or N527Y).

In some embodiments, the HSA variant (referred herein as mHSA) has the following substitutions: C34S. N503Q (SEQ ID NO: 13). In some embodiments, the HSA variant (referred herein as mHSA7) has the following substitutions C34S, N503Q, E505G and V547A (SEQ ID NO: 14). In some embodiments, the HSA variant has amino acids 25-609 and the following substitutions C58S and N527Q (SEQ ID NO: 15).

In some embodiments, the asparagine at position 503 and/or 527 of HSA, which may be deamidated and decrease half-life, can be removed by the N503Q substitution and/or the N527Q. In some embodiments, the cysteine C34 of HSA may be substituted to serine or alanine (S or A) to remove the free cysteine and minimize alternate disulfide-bond formation. In some embodiments, the half-life modulator is a modified version of the domain III (mHSA_dIII) of a modified HSA with the N503Q substitution and an additional terminal glycine. Such a modified version retains the HSA property of binding to FcRn and increased serum half-life.

In some embodiments, the half-life modulator is Fc domain of an antibody or a single chain constant fragment. In some embodiments, the half-life modulator comprises Fc regions of an immunoglobulin molecule (e.g. IgG). In some embodiments, the half-life modulator comprises at least 100 consecutive amino acids that are at least 70%, 80%, 85%, 90%, 91%, 92%, 93%, 94%, 95%, 96%, 97%, 98%, or 99% identical to a human Fc amino acid sequence. The Fc domain of an antibody has a natural capability to bind FcRn, resulting in an extended half-life. In some embodiments, the Fc domain of an antibody is engineered not to bind Fc(gamma)R In an exemplary embodiment, the Fc domain is engineered to substitute N297 with Q (N297Q variant). In some embodiments, the half-life modulator is a monomeric variant form of Fc (scFc). For example, the subset of IgG heavy chain which naturally dimerizes to form Fc is hinge-CH2-CH3. In some embodiments, the Fc domain is engineered to form a single chain by linking the hinge-CH2-CH3 with a flexible linker such as GGGGSGGGGSGGGGSGGGGS (SEQ ID NO: 16) to create a hinge-CH2-CH3-linker-hinge-CH2-CH3 chain. In an exemplary embodiment, the single chain Fc (scFc) is engineered to substitute N297 with Q and C220 with S (N297Q, C220S).

In some embodiments, the half-life modulator is a single chain variable fragment (scFv) of an antibody targeted to albumin or other circulating protein. In some embodiments, the half-life modulator comprises an amino acid sequence that is at least 70%, 80%, 85%, 90%, 91%, 92%, 93%, 94%, 95%, 96%, 97%, 98%, or 99% identical to scFv amino acid sequence directed to a specific antigen, such as, but not limited to, albumin. In some embodiments, the half-life modulator comprises at least 50, at least 100, at least 150, at least 200, at least 250 consecutive amino acids that are at least 70%, 80%, 85%, 90%, 91%, 92%, 93%, 94%, 95%, 96%, 97%, 98%, or 99% identical to a scFv amino acid sequence directed to a specific antigen, such as, but not limited to, albumin.

In some embodiments, the half-life modulator is transferrin such as human transferrin (Tf, SEQ ID NO: 17). In some embodiments, the half-life modulator comprises an amino acid sequence that is at least 70%, 80%, 85%, 90%, 91%, 92%, 93%, 94%, 95%, 96%, 97%, 98%, or 99% identical to human transferrin amino acid sequence. In some embodiments, the half-life modulator comprises at least 100, at least 200, at least 300, at least 400, at least 500, at least 600, at least 650 consecutive amino acids that are at least 70%, 80%, 85%, 90%, 91%, 92%, 93%, 94%, 95%, 96%, 97%, 98%, or 99% identical to a human transferrin amino acid sequence.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild type human alpha-fetoprotein amino acid sequence (AFP, SEQ ID NO: 18). In some embodiments, the half-life modulator comprises at least 100 consecutive amino acids that are about 70%, 80%, 85%, 90%, 91%, 92%, 93%, 94%, 95%, 96%, 97%, 98%, or 99% identical to wild type human alpha-fetoprotein (AFP) amino acid sequence. In some embodiments, the N-linked glycosylation site of the AFP is removed by the N251Q substitution.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical wild-type vitamin D-binding protein amino acid sequence (VDBP, SEQ ID NO: 19). In some embodiments, the half-life modulator comprises at least 100 consecutive amino acids that are about 70%, 80%, 85%, 90%, 91%, 92%, 93%, 94%, 95%, 96%, 97%, 98%, or 99% identical wild-type vitamin D-binding protein (VDBP) amino acid sequence. In some embodiments, the N-linked glycosylation site of the VDBP can be removed by the N288Q or N288T substitution.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild-type human transthyretin amino acid sequence (TTR, SEQ ID NO: 20). In some embodiments, the half-life modulator comprises at least 100 consecutive amino acids that are about 70%, 80%, 85%, 90%, 91%, 92%, 93%, 94%, 95%, 96%, 97%, 98%, or 99% identical to wild type human transthyretin (TTR) amino acid sequence. In some embodiments, the transthyretin is modified to remove the N118 N-glycosylation site. In some embodiments, the half-life modulator is a monomeric form of TTR.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to a PASylation amino acid sequence. PASylation are proline-, alanine-, and/or serine-rich sequences that mimic PEGylation (see WO/2008/155134). In some embodiments, the half-life modulator comprises at least 100 consecutive amino acids that are about 70%, 80%, 85%, 90%, 91%, 92%, 93%, 94%, 95%, 96%, 97%, 98%, or 99% identical to a PASylation amino acid sequence. PASylation are proline-, alanine-, and/or serine-rich sequences that mimic PEGylation. Polypeptide stretches of proline, alanine, and/or serine form semi-structured three-dimensional domains with large hydrodynamic radius, thereby reducing clearance of fusion proteins. In some embodiments, the PASylation amino acid sequence is about 200, 300, 400, 500 or 600 amino acids long. For example, the PASylation is a 20 times repeat of the amino acid sequence ASPAAPAPASPAAPAPSAPA (SEQ ID NO: 21).

In some embodiments, the half-life modulator comprises the attachment of polyethylene glycol (PEG) chain or chains to the fusion proteins through chemical attachment cither to the N- and/or C-terminus and/or to an amino acid side chain (e.g., PEG-maleimide attachment to cysteines). PEG chains form semi-structured three-dimensional domains with large hydrodynamic radius, thereby reducing clearance of fusion proteins.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to an albumin-binding domain human antibody (albudAb) amino acid sequence (SEQ ID NO: 22). In some embodiments, the half-life modulator comprises at least 100 consecutive amino acids that are about 70%, 80%, 85%, 90%, 91%, 92%, 93%, 94%, 95%, 96%, 97%, 98%, or 99% identical to an albumin-binding domain human antibody (albudAb) amino acid sequence. Albumin-binding domain antibodies can increase the fusion protein half-life by binding non-covalently to serum albumin (see WO2008/096158 which is incorporated herein by reference in its entirety). In some embodiments, the albumin-binding domain human antibody is engineered to remove the C-terminal arginine to remove the Lys-Arg Kex2 protease site.

Representative such half-life modulators include those recited in any one of SEQ ID NOs: 12-15, 17-22.

In some embodiments, the half-life modulators can be modified to substitute the cysteine residues to serine or alanine residues to reduce the ability to form disulfide bonds.

In some embodiments, the targeting domain and activator domain can be joined via a half-life modulator. Accordingly, the half-life modulator can have two termini, an N-terminus and a C-terminus. In some embodiments, the half-life modulator is joined at one terminus via a peptide bond to the targeting polypeptide domain and is joined at the other terminus via a peptide bond to the activator domain. In certain embodiments, the half-life modulator is joined at the N-terminus to the C-terminus of the targeting polypeptide domain and at the C-terminus to the N-terminus of the activator domain. In other embodiments, the half-life modulator is joined at the C-terminus to the targeting polypeptide domain and at the N-terminus to the activator domain. Yet, in other embodiments, the half-life modulator is joined at one of the termini of the bi-specific protein. For example, in some embodiments, the half-life modulator is joined at the C-terminus to the N-terminus of the activator domain. In other embodiments, the half-life modulator is joined at the N-terminus to the C-terminus of the targeting domain. In other embodiments, the half-life modulator can be joined at the N-terminus to the C-terminus of the activator domain. Yet in other embodiments, the half-life modulator can be joined at the N-terminus to the C-terminus of the targeting domain.

### Peptide Linkers

In some embodiments, the activator domain, half-life modulator, and targeting domain are linked by peptide linker (e.g., from 2 to 40, 2-50, 2-100 amino acid residues) such that upon target recognition and engagement by the targeting domain, the presentation of the activator domain is optimized for binding to and activation of extracellular receptors on the surface of cells that present the target at a given surface density (e.g.—5×10² molecules/1,000 Å2).

Targeted delivery of the activator domain for example IGF-1 for the activation of receptors on cells or tissues displaying a specific target requires appropriate presentation of both the activator domain and the targeting domain. In some embodiments, the flexibility of the linker is optimized for proper geometry of the engaged chimeric protein. Some of the principal determinants of the geometric constraints are the distances from the cell surface for the target and the receptor.

Additional optimization can be driven by the relative number of receptors and target molecules. At high ratios of Receptor:Target molecule, the engagement of both domains is reaction-limited. When the target molecule is more abundant than the receptor, the occupancy of both domains is diffusion-limited. Under the reaction-limit, optimal delivery of the activator domain is attained via short and rigid linkers. Under the diffusion limit, long and flexible linkers allow t he activator domain to access a larger surface area. For cells with complex shapes (i.e.—bodies and neuronal processes) and receptor distributions, appropriate design of linker flexibility can enable precise targeting to sub-cellular regions.

In some embodiments, the peptide linker is present at the N-terminus, at the C-terminus or at both the N-terminus and the C-terminus of the half-life modulator at one or both ends. Suitable short connector polypeptides for use at the N-terminal end of the linker include, for example, dipeptides such as -Gly-Ser- (GS), -Gly-Ala- (GA) and -Ala-Ser- (AS). Suitable peptide linkers for use at the C-terminal end of the linker include, for example, dipeptides such as -Leu-Gln- (LQ) and -Thr-Gly- (TG). In some embodiments, the peptide linkers are longer than 2 amino acids. For example, the peptide linkers are 5, 6, 7, 8, 9, 10, 11, 12, 13, 14, 15, 16, 17, 18, 19 or 20 amino acids long or longer. In some embodiments, the peptide linkers are 20 or more 30 or more, 40 or more, 50 or more, 60 or more, 70 or more, 80 or more, 90 or more, 100 or more amino acids long. Preferably, such peptide linkers are flexible (for example glycine-rich) or structured (e.g., alpha-helix rich). In some embodiments, the linker comprises or consist of amino acids -Gly-Ser-Gly-Gly-Gly-Ser-Gly (SEQ ID NO: 23).

It will be apparent that elements in addition to those described above may optionally be included in the proteins provided herein. Such elements may be present for a variety of purposes, including to facilitate expression, preparation or purification of the bi-specific fusion protein, or to perform targeting functions.

### Representative Chimeric Protein

In some embodiments, a representative bi-specific fusion proteins comprise (from N-terminal to C-terminal):
(a) a targeting polypeptide domain comprising or consisting of a non-internalizing human annexin V variant (e.g., comprising or consisting of amino acids 2-320 of wt human Annexin 5 and a substitution at C316, R63, K40, K101, E138, D139, N160);
(b) a linker peptide (e.g., -Gly-Ser-Gly-Gly-Gly-Ser-Gly) (SEQ ID NO: 23);
(c) a half-life modulator (e.g., HSA variant comprising or consisting of amino acids 25-609 of wt human HSA and comprising substitution at C58 and N527);
(d) linker peptide (e.g., -Gly-Ser-Gly-Gly-Gly-Ser-Gly) (SEQ ID NO: 23);
(e) an activator domain comprising or consisting of an IGF-1 variant (e.g., comprising a substitution at E3 and Y31.

In some embodiments, the chimeric protein comprises or consists of IGF1(E3R/Y31A)_lk7_HSA25-609(C58S/N527Q)_lk7_AnxV2-320(R63A/K70A/K101A/E138A/D139G/N160A/C316A) (also referred herein as scp776). In some embodiments, the chimeric protein has an amino acid sequence as set forth in SEQ ID NO: 24.

In some embodiments, the chimeric protein comprises or consists of IGF1(E3R/Y31A)_lk7_HSA25-609(C58S/N527Q)_lk7_AnxV2-320(R63A/K70A/K101A/E138A/D139G/N160A/C316S). In some embodiments, the chimeric protein has an amino acid sequence as set forth in SEQ ID NO: 25.

### Nucleic Acids

Provided herein are polynucleotides encoding the chimeric proteins that may be in the form of RNA or in the form of DNA, which DNA includes cDNA and synthetic DNA. The DNA may be double-stranded or single-stranded. The coding sequences that encode the variants of the present disclosure may vary as a result of the redundancy or degeneracy of the genetic code.

### Pharmaceutical Compositions—Methods of Treatment

Methods of treatment and pharmaceutical compositions for use in the treatment of neurodegenerative disorders are provided.

In some embodiments, methods of treatment and/or neuroprotection for chronic neurodegenerative diseases (e.g., Alzheimer, Parkinson's Disease, ALS). In some embodiments, methods of treatment of Alzheimer disease are provided. In some embodiments, methods of treatment of Parkinson disease are provided. In some embodiments, methods of treatment of Amyotrophic Lateral Sclerosis are provided. In some embodiments, the method results in at least one or more of the following: improvement in neurological function, improvement in mental function, and increased survival rate. Measures of neurological function can be assessed with Unified Parkinson's Disease Rating Scale (UPDRS), the Sandoz Clinical Assessment-Geriatric (SCAG) scale, and ALS functional rating score, or variations of these scores.

Pharmaceutical compositions comprising a therapeutically effective amount of at least one chimeric protein as described herein, together with at least one physiologically acceptable carrier, are provided. Such compositions may be used for treating patients who are suffering from, or at risk for, tissue damage, in order to prevent tissue damage, or to repair or regenerate damaged tissue, for example nervous system tissue. The chimeric proteins and the pharmaceutical compositions described herein can have a neuroprotective action and prevent or halt neuronal death. Such patients include, for example, patients who have experienced neurodegenerative disorders. If desired, other active ingredients may also be included within the pharmaceutical composition, such as stem cells or other agents that facilitate repair of damaged tissue.

A “patient” or a “subject” is a mammal, preferably a human. The term “treating” (or “treat” or “treatment”) means slowing, reducing, or reversing the progression or severity of a symptom, disorder, condition, or disease.

The term “therapeutically effective amount” refers to the amount or dose of chimeric proteins described herein which, upon single or multiple dose administration to a patient, provides the desired treatment.

In some embodiments, the compositions or chimeric proteins described herein localizes to the central nervous system (CNS) tissue(s) subjected to ischemic injury whether by traversing the blood brain barrier (BBB) or due to permeabilization of the blood brain barrier.

In some embodiments, the compositions or chimeric proteins described herein delivers pro-survival signals to neurons and glia of the subject.

In some embodiments, the pharmaceutical compositions or chimeric proteins described herein accelerates repair processes to the blood brain barrier of the subject in need thereof. In some embodiments, the pharmaceutical compositions or chimeric proteins described herein protects the blood brain barrier of the subject in need thereof.

In some embodiments, the pharmaceutical compositions or the chimeric protein can be administered to a subject in need thereof to protect or treat tissue or organ degeneration. For example, the pharmaceutical compositions can be used to treat neurodegenerative disorders such as Alzheimer's Disease (AD), Parkinson's Disease (PD).

In some embodiments, the pharmaceutical compositions can be administered in combination with or as a co-medication to existing treatments known in the art.

As used herein, the term “physiologically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the bi-specific fusion protein is administered. Physiologically acceptable carriers can be sterile liquids, such as water and oils, including those of petroleum, animal, vegetable or synthetic origin (e.g., peanut oil, soybean oil, mineral oil, or sesame oil). Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water and ethanol. The pharmaceutical composition, if desired, can also contain minor amounts of wetting or emulsifying agents, or pH buffering agents.

In some embodiments, the pharmaceutical composition of the disclosure is a liquid composition. In some embodiments, the pharmaceutical composition of the disclosure is formulated for intravenous injection, intrathecal injection, or intranigral injection.

### IV Formulations

In some embodiments, the pharmaceutical composition is a liquid formulation formulated for intravenous (IV) injection. In some embodiments, the composition is formulated for IV bolus injection.

In some embodiments, the pH of the pharmaceutical composition is from about 7 to about 8, for example about 7.5. In some embodiments, the pharmaceutical composition comprises in an alkalinizing agent, such as tromethamine or dibasic sodium phosphate. In some embodiments, the alkalinizing can be at a concentration of about 10 to about 50 mM, for example about 20 mM. In some embodiments, the pharmaceutical composition comprises a surfactant. In some embodiments, the surfactant is a non-ionic surfactant such as polysorbate 80 or polysorbate 20 may be present in a concentration of about 0.01%, 0.02%. 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% (w/v) or any concentration therebetween. In some embodiments, the pharmaceutical composition comprises sucrose. In some embodiments, the sucrose may be present in a concentration of about 1%, 2%. 3%, 4%, 5%, 6%, 7%, 8%, 9%, 10% (w/v) or any concentration therebetween.

In some embodiments, the pharmaceutical composition comprising the chimeric protein comprises alkalinizing agent, a surfactant, sucrose or a combination thereof.

The pharmaceutical compositions of the disclosure may contain pharmaceutically acceptable auxiliary substances as required to approximate physiological conditions, such as pH adjusting and buffering agents, tonicity adjusting agents, wetting agents, detergents, preservatives and the like.

In some embodiments, the pharmaceutical composition comprises from about 1 to about 100 g/l chimeric protein, from about 10 mM to about 50 mM tromethamine, from about 2 to about 15% (w/v) sucrose, from about 0.001% to about 0.04% (w/v) Polysorbate 80, at pH 7.5.

In some embodiments, the chimeric protein (from about 1 to about 100 g/l) is formulated in about 20 mM tromethamine, about 7.5% sucrose, and about 0.02% polysorbate 80 at pH 7.5 (via addition of HCl). In some embodiments, about 20 mg/ml of the chimeric protein is formulated in about 20 mM tromethamine, about 7.5% sucrose, and about 0.02% polysorbate 80 at pH 7.5 (via addition of HCl).

In some embodiments, the liquid composition is sterilized by conventional sterilization techniques, or sterile filtered. In some embodiments, the liquid composition is in a vial.

In some embodiments, the pharmaceutical composition is administered intravenously or intraarterially to a subject in need thereof via bolus injection. A bolus injection comprises, e.g., fast intravenous injection, for example less than 10 seconds (or less than 20, 30, 40, 50, 60 second), or intravenous infusion over less than approximately 3 minutes, 4 minutes, 5 minutes, 6 minutes, 7 minutes, 8 minutes, 9 minutes or 10 minutes. In some embodiments, the pharmaceutical composition is administered intravenously to a subject in need thereof. In some embodiments, the composition is administered as an I.V. push. In some embodiments, the administration is over 5 min. or less, 4 min. or less, 3 min. of less, 2 min. or less, 1 min or less (for example 50 s, 40 s, 30 s, 20 s or any administration time therebetween). In other embodiments, the composition is administered as a slow I.V. injection.

In some embodiments, the pharmaceutical composition is administered intravenously to a subject using a syringe injection pump at a rate of 0.5-25 mL/min.

In some embodiments, the pharmaceutical composition is administered intraarterially to a subject using a syringe injection pump at a rate of 0.05-10 mL/min.

### Intrathecal Formulations

In some embodiments, the pharmaceutical composition is a liquid formulation formulated for intrathecal (IT) administration. In some embodiments, the pharmaceutical composition is administered intrathecally to a subject in need thereof using techniques known in the art including, but not limited to, implantable pumps and manual syringes/needles.

In some embodiments, the intrathecal formulation is stable under accelerated conditions (RT) and under refrigerated conditions (4° C.).

In some embodiments, the pharmaceutical composition is a liquid formulation formulated for intrathecal (IT) administration.

In some embodiments, the pH of the pharmaceutical composition is from about 4.5 to about 6.5, from about 4.5 to about 5, from about 5 to about 5.5, from about 5.5 to about 6, from about 6 to about 6.5, about 4.5, about 5, about 5.5, about 6, about 6.6. In some embodiments, the pharmaceutical composition comprises acetate, phosphate, citrate, histidine, for example at a concentration of 10 to 50 mM. In some embodiments, the pharmaceutical composition comprises acetate. In some embodiments, the citrate can be at a concentration of about 10 to about 50 mM, for example about 20 mM. In some embodiments, the pharmaceutical composition comprises sucrose. In some embodiments, the sucrose may be present in a concentration of about 1%, 2%. 3%, 4%, 5%, 6%, 7%, 8%, 9%, 10% or any concentration therebetween (w/v).

In some embodiments, the pharmaceutical composition comprising the chimeric protein comprises citrate, sucrose or a combination thereof.

The pharmaceutical compositions of the disclosure may contain pharmaceutically acceptable auxiliary substances as required to approximate physiological conditions, such as pH adjusting and buffering agents, tonicity adjusting agents, wetting agents, detergents, preservatives and the like.

In some embodiments, the pharmaceutical composition is formulated for intrathecal delivery. In some embodiments, the intrathecal formulation comprises less than 2% (w/v) sucrose, less than 1.5% (w/v) sucrose, less than 1% (w/v) sucrose, less 0.5% (w/v) sucrose, less than 0.1% (w/v) sucrose, less than 0.01% (w/v) sucrose. In some embodiments, the intrathecal formulation is free of sucrose.

In some embodiments, the intrathecal formulation comprises from about 1 to about 25 g/l chimeric protein or any value therebetween.

In some embodiments, the intrathecal formulation has a pH of from about 5 to about 7, for example about 5, about 5.5, about 6, about 6.5, about 7.

In some embodiments, the intrathecal formulation comprises from about 100 mM to about 150 mM sodium chloride and sterile water. In some embodiments, the intrathecal formulation is a preservative-free formulation. In some embodiments, the intrathecal formulation is sterile.

In some embodiments, the intrathecal formulation comprises excipients that are such as, but not limited, to histidine, histidine monohydrochloride, histidine hydrochloride, methionine, succinic acid, trehalose dihydrate, polysorbate 20, and polysorbate 80.

In some embodiments, the intrathecal formulation has an osmolality of about 45-140 mOsmol/kg, or any value therebetween, for example 45-75 mOsmol/kg, 74-110 mOsmol/kg or 110-140 mOsmol/kg.

In some embodiments, the intrathecal formulation comprises a therapeutically effective amount of the chimeric protein, about 20 mM sodium citrate, about 122 mM sodium chloride and has a pH of about 6.5. In some embodiments, the intrathecal formulation consists essentially of a therapeutically effective amount of the chimeric protein, about 20 mM sodium citrate, about 122 mM sodium chloride and has a pH of about 6.5. In some embodiments, the intrathecal formulation consists of a therapeutically effective amount of the chimeric protein, about 20 mM sodium citrate, about 122 mM sodium chloride and has a pH of about 6.5.

In some embodiments, the intrathecal formulation is administered using an implantable pump (i.e. “pain pump”) or manually using an appropriate needle affixed to a syringe.

In some embodiments, the pharmaceutical composition comprises from about 1 to about 25 g/l chimeric protein, from about 2 to about 15% (w/v) sucrose, at pH 5.

In some embodiments, the chimeric protein (from about 1 to about 10 g/l) is formulated in about 20 mM citrate, about 7.5% sucrose, at pH 5. In some embodiments, about 5 mg/ml of the chimeric protein is formulated in about 20 mM citrate, about 7.5% sucrose, at pH 5.

In some embodiments, the liquid composition is sterilized by conventional sterilization techniques, or sterile filtered. In some embodiments, the liquid composition is in a vial.

### Intranigral Formulation

In some embodiments, the pharmaceutical composition is formulated for intranigral delivery. In some embodiments, the intranigral formulation comprises less than 2% (w/v) sucrose, less than 1.5% (w/v) sucrose, less than 1% (w/v) sucrose, less 0.5% (w/v) sucrose, less than 0.1% (w/v) sucrose, less than 0.01% (w/v) sucrose. In some embodiments, the intranigral formulation is free of sucrose.

In some embodiments, the intranigral formulation comprises from about 1 to about 25 g/l chimeric protein or any value therebetween.

In some embodiments, the intranigral formulation has a pH of from about 5 to about 7, for example about 5, about 5.5, about 6, about 6.5, about 7.

In some embodiments, the intranigral formulation comprises from about 100 mM to about 150 mM sodium chloride and sterile water. In some embodiments, the intranigral formulation is a preservative-free formulation. In some embodiments, the intranigral formulation is sterile.

In some embodiments, the intranigral formulation comprises excipients that are such as, but not limited, to histidine, histidine monohydrochloride, histidine hydrochloride, methionine, succinic acid, trehalose dihydrate, polysorbate 20, and polysorbate 80.

In some embodiments, the intranigral formulation comprises a therapeutically effective amount of the chimeric protein, about 20 mM sodium citrate, about 122 mM sodium chloride and has a pH of about 6.5. In some embodiments, the intranigral formulation consists essentially of a therapeutically effective amount of the chimeric protein, about 20 mM sodium citrate, about 122 mM sodium chloride and has a pH of about 6.5. In some embodiments, the intranigral formulation consists of a therapeutically effective amount of the chimeric protein, about 20 mM sodium citrate, about 122 mM sodium chloride and has a pH of about 6.5.

In some embodiments, the intranigral formulation has an osmolality of about 45-140 mOsmol/kg, or any value therebetween, for example 45-75 mOsmol/kg, 74-110 mOsmol/kg or 110-140 mOsmol/kg.

### Dosage

In some embodiments, a therapeutically effective amount generally is in the range of about 0.01 mg/kg to about 100.0 mg/kg per dose. In some embodiments, a therapeutically effective amount of a protein disclosed herein can be, e.g., about 0.01 mg/kg to about 0.1 mg/kg per dose, 0.01 mg/kg to about 1 mg/kg per dose, 0.01 mg/kg to about 10 mg/kg per dose, 0.01 mg/kg to about 100.0 mg/kg per dose, 0.1 mg/kg to about 1 mg/kg per dose, 0.1 mg/kg to about 10 mg/kg per dose, 0.1 mg/kg to about 100 mg/kg per dose, 1 mg/kg to about 100 mg/kg per dose or 10 mg/kg to about 100 mg/kg per dose.

In some embodiments, a therapeutically effective amount generally is in the range of about 0.01 mg/kg to about 20.0 mg/kg per dose. In some embodiments, a therapeutically effective amount cam be range from about 0.01 mg/kg to about 10.0 mg/kg per dose.

In some embodiments, a therapeutically effective amount of a protein disclosed herein can be from about 0.01 mg/kg to about 0.02 mg/kg per dose, about 0.01 mg/kg to about 0.03 mg/kg per dose, about 0.01 mg/kg to about 0.04 mg/kg per dose, about 0.01 mg/kg to about 0.05 mg/kg per dose, about 0.01 mg/kg to about 0.06 mg/kg per dose, about 0.01 mg/kg to about 0.07 mg/kg per dose, about 0.01 mg/kg to about 0.08 mg/kg per dose, about 0.01 mg/kg to about 0.09 mg/kg per dose, about 0.01 mg/kg to about 0.1 mg/kg per dose, about 0.01 mg/kg to about 0.2 mg/kg per dose, about 0.01 mg/kg to about 0.3 mg/kg per dose, about 0.01 mg/kg to about 0.4 mg/kg per dose, about 0.01 mg/kg to about 0.5 mg/kg per dose, about 0.01 mg/kg to about 0.6 mg/kg per dose, about 0.01 mg/kg to about 0.7 mg/kg per dose, about 0.01 mg/kg to about 0.8 mg/kg per dose, about 0.01 mg/kg to about 0.9 mg/kg per dose, about 0.01 mg/kg to about 1 mg/kg per dose, about 0.01 mg/kg to about 2 mg/kg per dose, about 0.01 mg/kg to about 3 mg/kg per dose, about 0.01 mg/kg to about 4 mg/kg per dose, about 0.01 mg/kg to about 5 mg/kg per dose, about 0.01 mg/kg to about 6 mg/kg per dose, about 0.01 mg/kg to about 7 mg/kg per dose, about 0.01 mg/kg to about 8 mg/kg per dose, about 0.01 mg/kg to about 9 mg/kg per dose, about 0.01 mg/kg to about 10 mg/kg per dose, about 0.01 mg/kg to about 11 mg/kg per dose, about 0.01 mg/kg to about 12 mg/kg per dose, about 0.01 mg/kg to about 13 mg/kg per dose, about 0.01 mg/kg to about 14 mg/kg per dose, about 0.01 mg/kg to about 15 mg/kg per dose, about 0.01 mg/kg to about 16 mg/kg per dose, about 0.01 mg/kg to about 17 mg/kg per dose, about 0.01 mg/kg to about 18 mg/kg per dose, about 0.01 mg/kg to about 19 mg/kg per dose, about 0.01 mg/kg to about 20 mg/kg per dose.

In some embodiments, a therapeutically effective amount of a protein disclosed herein can be from about 0.01 mg/kg to about 0.05 mg/kg per dose, about 0.05 mg/kg to about 0.1 mg/kg per dose, about 0.1 mg/kg to about 0.5 mg/kg per dose, about 0.05 mg/kg to about 1 mg/kg per dose, about 1 mg/kg to about 2 mg/kg per dose, about 2 mg/kg to about 3 mg/kg per dose, about 3 mg/kg to about 4 mg/kg per dose, about 4 mg/kg to about 5 mg/kg per dose, about 5 mg/kg to about 6 mg/kg per dose, about 6 mg/kg to about 7 mg/kg per dose, about 7 mg/kg to about 8 mg/kg per dose, about 8 mg/kg to about 9 mg/kg per dose, about 9 mg/kg to about 10 mg/kg per dose, about 10 mg/kg to about 11 mg/kg per dose, about 11 mg/kg to about 12 mg/kg per dose, about 12 mg/kg to about 13 mg/kg per dose, about 13 mg/kg to about 14 mg/kg per dose, about 14 mg/kg to about 15 mg/kg per dose, about 15 mg/kg to about 16 mg/kg per dose, about 16 mg/kg to about 17 mg/kg per dose, 17 mg/kg to about 18 mg/kg per dose, about 18 mg/kg to about 19 mg/kg per dose, about 19 mg/kg to about 20 mg/kg per dose.

In some embodiments, a therapeutically effective amount of a protein disclosed herein can be about 0.01 mg/kg per dose, about 0.02 mg/kg per dose, about 0.03 mg/kg per dose, about 0.04 mg/kg per dose, about 0.05 mg/kg per dose, about 0.06 mg/kg per dose, about 0.07 mg/kg per dose, about 0.08 mg/kg per dose, about 0.09 mg/kg per dose, about 0.1 mg/kg per dose, about 0.2 mg/kg per dose, about 0.3 mg/kg per dose, about 0.4 mg/kg per dose, about 0.5 mg/kg per dose, about 0.6 mg/kg per dose, about 0.7 mg/kg per dose, about 0.8 mg/kg per dose, about 0.9 mg/kg per dose, about 1 mg/kg per dose, about 1.1 mg/kg per dose, about 1.2 mg/kg per dose, about 1.3 mg/kg per dose, about 1.4 mg/kg per dose, about 1.5 mg/kg per dose, about 1.6 mg/kg per dose, about 1.7 mg/kg per dose, about 1.8 mg/kg per dose, about 1.9 mg/kg per dose, about 2 mg/kg per dose, about 2.1 mg/kg per dose, about 2.2 mg/kg per dose, about 2.3 mg/kg per dose, about 2.4 mg/kg per dose, about 2.5 mg/kg per dose, about 2.6 mg/kg per dose, about 2.7 mg/kg per dose, about 2.8 mg/kg per dose, about 2.9 mg/kg per dose, about 3 mg/kg per dose, about 3.1 mg/kg per dose, about 3.2 mg/kg per dose, about 3.3 mg/kg per dose, about 3.4 mg/kg per dose, about 3.5 mg/kg per dose, about 3.6 mg/kg per dose, about 3.7 mg/kg per dose, about 3.8 mg/kg per dose, about 3.9 mg/kg per dose, about 4 mg/kg per dose, about 4.1 mg/kg per dose, about 4.2 mg/kg per dose, about 4.3 mg/kg per dose, about 4.4 mg/kg per dose, about 4.5 mg/kg per dose, about 4.6 mg/kg per dose, about 4.7 mg/kg per dose, about 4.8 mg/kg per dose, about 4.9 mg/kg per dose, about 5 mg/kg per dose, about 5.1 mg/kg per dose, about 5.2 mg/kg per dose, about 5.3 mg/kg per dose, about 5.4 mg/kg per dose, about 5.5 mg/kg per dose, about 5.6 mg/kg per dose, about 5.7 mg/kg per dose, about 5.8 mg/kg per dose, about 5.9 mg/kg per dose, about 6 mg/kg per dose, about 6.1 mg/kg per dose, about 6.2 mg/kg per dose, about 6.3 mg/kg per dose, about 6.4 mg/kg per dose, about 6.5 mg/kg per dose, about 6.6 mg/kg per dose, about 6.7 mg/kg per dose, about 6.8 mg/kg per dose, about 6.9 mg/kg per dose, about 7 mg/kg per dose, about 7.1 mg/kg per dose, about 7.2 mg/kg per dose, about 7.3 mg/kg per dose, about 7.4 mg/kg per dose, about 7.5 mg/kg per dose, about 7.6 mg/kg per dose, about 7.7 mg/kg per dose, about 7.8 mg/kg per dose, about 7.9 mg/kg per dose, about 8 mg/kg per dose, about 8.1 mg/kg per dose, about 8.2 mg/kg per dose, about 8.3 mg/kg per dose, about 8.4 mg/kg per dose, about 8.5 mg/kg per dose, about 8.6 mg/kg per dose, about 8.7 mg/kg per dose, about 8.8 mg/kg per dose, about 8.9 mg/kg per dose, about 9 mg/kg per dose, about 9.1 mg/kg per dose, about 9.2 mg/kg per dose, about 9.3 mg/kg per dose, about 9.4 mg/kg per dose, about 9.5 mg/kg per dose, about 9.6 mg/kg per dose, about 9.7 mg/kg per dose, about 9.8 mg/kg per dose, about 9.9 mg/kg per dose, about 10 mg/kg per dose, about 11 mg/kg per dose, about 12 mg/kg per dose, about 13 mg/kg per dose, about 14 mg/kg per dose, about 15 mg/kg per dose, about 16 mg/kg per dose, about 17 mg/kg per dose, about 18 mg/kg per dose, about 19 mg/kg per dose, about 20 mg/kg per dose.

In some embodiments, a therapeutically effective amount generally is in the range of about 1 mg/kg to about 10.0 mg/kg per dose. In some embodiments, an effective amount of a protein disclosed herein can be, e.g., about 1 mg/kg to about 10 mg/kg per dose, 1 mg/kg to about 9 mg/kg per dose, 1 mg/kg to about 8 mg/kg per dose, 1 mg/kg to about 7 mg/kg per dose, 1 mg/kg to about 6 mg/kg per dose, 1 mg/kg to about 5 mg/kg per dose, 1 mg/kg to about 4 mg/kg per dose, 1 mg/kg to about 3 mg/kg per dose or 1 mg/kg to about 2 mg/kg per dose.

Dosing can be single dosage or cumulative (serial dosing), and can be readily determined by one skilled in the art. For instance, treatment of a nervous system disorder may comprise a one-time administration of an effective dose of the pharmaceutical composition disclosed herein. As a non-limiting example, an effective dose of the composition disclosed herein can be administered once to a patient, e.g., as a single injection or bolus. Alternatively, treatment of a nervous system disorder may comprise multiple administrations of an effective dose of the pharmaceutical composition disclosed herein carried out over a range of time periods, such as, e.g., four times daily, three times daily, twice daily, daily, once every few days, weekly, monthly or yearly. As a non-limiting example, a combination disclosed herein can be administered once or twice weekly to a patient. The timing of administration can be determined upon such factors as the severity of the patient's symptoms. For example, an effective dose of the composition disclosed herein can be administered to a patient once a month for an indefinite period of time, or until the mammal no longer requires therapy.

In some embodiments, a therapeutically effective amount generally is in the range of about 0.01 mg/kg to about 200.0 mg/kg per day. In some embodiments, an effective amount of a protein disclosed herein can be, e.g., .g., about 0.01 mg/kg to about 0.1 mg/kg per day, 0.01 mg/kg to about 1 mg/kg per day, 0.01 mg/kg to about 10 mg/kg per day, 0.01 mg/kg to about 100.0 mg/kg per day, 0.01 mg/kg to about 200.0 mg/kg per day, 0.1 mg/kg to about 1 mg/kg per day, 0.1 mg/kg to about 10 mg/kg per day, 0.1 mg/kg to about 100 mg/kg per day, 0.1 mg/kg to about 200 mg/kg per day, 1 mg/kg to about 100 mg/kg per day, 1 mg/kg to about 200 mg/kg per day, 10 mg/kg to about 100 mg/kg per day or 10 mg/kg to about 100 mg/kg per day.

In some embodiments, a therapeutically effective amount generally is in the range of about 0.01 mg/kg to about 20.0 mg/kg per day. In some embodiments, a therapeutically effective amount generally is in the range of about 0.01 mg/kg to about 10.0 mg/kg per day. In some embodiments, a therapeutically effective amount generally is in the range of about 1 mg/kg to about 20.0 mg/kg per day. In some embodiments, a therapeutically effective amount generally is in the range of about 1 mg/kg to about 10.0 mg/kg per day. In some embodiments, an effective amount of a protein disclosed herein can be, e.g., about 1 mg/kg to about 10 mg/kg per day, 1 mg/kg to about 9 mg/kg per day, 1 mg/kg to about 8 mg/kg per day, 1 mg/kg to about 7 mg/kg per day, 1 mg/kg to about 6 mg/kg per day, 1 mg/kg to about 5 mg/kg per day, 1 mg/kg to about 4 mg/kg per day, 1 mg/kg to about 3 mg/kg per day or 1 mg/kg to about 2 mg/kg per day.

In some embodiments, a therapeutically effective amount of a chimeric protein disclosed herein can be from about 0.01 mg/kg to about 0.02 mg/kg per day, about 0.01 mg/kg to about 0.03 mg/kg per day, about 0.01 mg/kg to about 0.04 mg/kg per day, about 0.01 mg/kg to about 0.05 mg/kg per day, about 0.01 mg/kg to about 0.06 mg/kg per day, about 0.01 mg/kg to about 0.07 mg/kg per day, about 0.01 mg/kg to about 0.08 mg/kg per day, about 0.01 mg/kg to about 0.09 mg/kg per day, about 0.01 mg/kg to about 0.1 mg/kg per day, about 0.01 mg/kg to about 0.2 mg/kg per day, about 0.01 mg/kg to about 0.3 mg/kg per day, about 0.01 mg/kg to about 0.4 mg/kg per day, about 0.01 mg/kg to about 0.5 mg/kg per day, about 0.01 mg/kg to about 0.6 mg/kg per day, about 0.01 mg/kg to about 0.7 mg/kg per day, about 0.01 mg/kg to about 0.8 mg/kg per day, about 0.01 mg/kg to about 0.9 mg/kg per day, about 0.01 mg/kg to about 1 mg/kg per day, about 0.01 mg/kg to about 2 mg/kg per day, about 0.01 mg/kg to about 3 mg/kg per day, about 0.01 mg/kg to about 4 mg/kg per day, about 0.01 mg/kg to about 5 mg/kg per day, about 0.01 mg/kg to about 6 mg/kg per day, about 0.01 mg/kg to about 7 mg/kg per day, about 0.01 mg/kg to about 8 mg/kg per day, about 0.01 mg/kg to about 9 mg/kg per day, about 0.01 mg/kg to about 10 mg/kg per day, about 0.01 mg/kg to about 11 mg/kg per day, about 0.01 mg/kg to about 12 mg/kg per day, about 0.01 mg/kg to about 13 mg/kg per day, about 0.01 mg/kg to about 14 mg/kg per day, about 0.01 mg/kg to about 15 mg/kg per day, about 0.01 mg/kg to about 16 mg/kg per day, about 0.01 mg/kg to about 17 mg/kg per day, about 0.01 mg/kg to about 18 mg/kg per day, about 0.01 mg/kg to about 19 mg/kg per day, or about 0.01 mg/kg to about 20 mg/kg per day.

In some embodiments, a therapeutically effective amount of a chimeric protein disclosed herein can be from about 0.01 mg/kg to about 0.05 mg/kg per day, about 0.05 mg/kg to about 0.1 mg/kg per day, about 0.1 mg/kg to about 0.5 mg/kg per day, about 0.05 mg/kg to about 1 mg/kg per day, about 1 mg/kg to about 2 mg/kg per day, about 2 mg/kg to about 3 mg/kg per day, about 3 mg/kg to about 4 mg/kg per day, about 4 mg/kg to about 5 mg/kg per day, about 5 mg/kg to about 6 mg/kg per day, about 6 mg/kg to about 7 mg/kg per day, about 7 mg/kg to about 8 mg/kg per day, about 8 mg/kg to about 9 mg/kg per day, about 9 mg/kg to about 10 mg/kg per day, about 10 mg/kg to about 11 mg/kg per day, about 11 mg/kg to about 12 mg/kg per day, about 12 mg/kg to about 13 mg/kg per day, about 13 mg/kg to about 14 mg/kg per day, about 14 mg/kg to about 15 mg/kg per day, about 15 mg/kg to about 16 mg/kg per day, about 16 mg/kg to about 17 mg/kg per day, 17 mg/kg to about 18 mg/kg per day, about 18 mg/kg to about 19 mg/kg per day, or about 19 mg/kg to about 20 mg/kg per day.

In some embodiments, a therapeutically effective amount of a chimeric protein disclosed herein can be about 0.01 mg/kg per day, about 0.02 mg/kg per day, about 0.03 mg/kg per day, about 0.04 mg/kg per day, about 0.05 mg/kg per day, about 0.06 mg/kg per day, about 0.07 mg/kg per day, about 0.08 mg/kg per day, about 0.09 mg/kg per day, about 0.1 mg/kg per day, about 0.2 mg/kg per day, about 0.3 mg/kg per day, about 0.4 mg/kg per day, about 0.5 mg/kg per day, about 0.6 mg/kg per day, about 0.7 mg/kg per day, about 0.8 mg/kg per day, about 0.9 mg/kg per day, about 1 mg/kg per day, about 1.1 mg/kg per day, about 1.2 mg/kg per day, about 1.3 mg/kg per day, about 1.4 mg/kg per day, about 1.5 mg/kg per day, about 1.6 mg/kg per day, about 1.7 mg/kg per day, about 1.8 mg/kg per day, about 1.9 mg/kg per day, about 2 mg/kg per day, about 2.1 mg/kg per day, about 2.2 mg/kg per day, about 2.3 mg/kg per day, about 2.4 mg/kg per day, about 2.5 mg/kg per day, about 2.6 mg/kg per day, about 2.7 mg/kg per day, about 2.8 mg/kg per day, about 2.9 mg/kg per day, about 3 mg/kg per day, about 3.1 mg/kg per day, about 3.2 mg/kg per day, about 3.3 mg/kg per day, about 3.4 mg/kg per day, about 3.5 mg/kg per day, about 3.6 mg/kg per day, about 3.7 mg/kg per day, about 3.8 mg/kg per day, about 3.9 mg/kg per day, about 4 mg/kg per day, about 4.1 mg/kg per day, about 4.2 mg/kg per day, about 4.3 mg/kg per day, about 4.4 mg/kg per day, about 4.5 mg/kg per day, about 4.6 mg/kg per day, about 4.7 mg/kg per day, about 4.8 mg/kg per day, about 4.9 mg/kg per day, about 5 mg/kg per day, about 5.1 mg/kg per day, about 5.2 mg/kg per day, about 5.3 mg/kg per day, about 5.4 mg/kg per day, about 5.5 mg/kg per day, about 5.6 mg/kg per day, about 5.7 mg/kg per day, about 5.8 mg/kg per day, about 5.9 mg/kg per day, about 6 mg/kg per day, about 6.1 mg/kg per day, about 6.2 mg/kg per day, about 6.3 mg/kg per day, about 6.4 mg/kg per day, about 6.5 mg/kg per day, about 6.6 mg/kg per day, about 6.7 mg/kg per day, about 6.8 mg/kg per day, about 6.9 mg/kg per day, about 7 mg/kg per day, about 7.1 mg/kg per day, about 7.2 mg/kg per day, about 7.3 mg/kg per day, about 7.4 mg/kg per day, about 7.5 mg/kg per day, about 7.6 mg/kg per day, about 7.7 mg/kg per day, about 7.8 mg/kg per day, about 7.9 mg/kg per day, about 8 mg/kg per day, about 8.1 mg/kg per day, about 8.2 mg/kg per day, about 8.3 mg/kg per day, about 8.4 mg/kg per day, about 8.5 mg/kg per day, about 8.6 mg/kg per day, about 8.7 mg/kg per day, about 8.8 mg/kg per day, about 8.9 mg/kg per day, about 9 mg/kg per day, about 9.1 mg/kg per day, about 9.2 mg/kg per day, about 9.3 mg/kg per day, about 9.4 mg/kg per day, about 9.5 mg/kg per day, about 9.6 mg/kg per day, about 9.7 mg/kg per day, about 9.8 mg/kg per day, about 9.9 mg/kg per day, about 10 mg/kg per day, about 11 mg/kg per day, about 12 mg/kg per day, about 13 mg/kg per day, about 14 mg/kg per day, about 15 mg/kg per day, about 16 mg/kg per day, about 17 mg/kg per day, about 18 mg/kg per day, about 19 mg/kg per day, or about 20 mg/kg per day.

In some embodiments, the effective dose is administered once a day to the subject in need thereof over a period of 2 days, 3 days, 4 days, 5 days, 6 days, 7 days, 8 days, 9 days, 10 days, 11 days, 12 days, 13 days, 14 days or more. In some embodiments, the effective dose is administered day to the subject having an acute CNS injury over a period of a minimum 2 days to a period of 14 days, for example 4, 5, 6, 7 days.

In some embodiments, the effective dose is administered once a day to the subject having neurovegetative disease over a period of a minimum 2 days to at least 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 11 years, 12, years, 13 years, 14 years, 15 years, 16, years, 17 years, 18 years, 19 years or 20 years.

In some embodiments, the effective amount is administered once a day (or every 24 hours) to the subject in need thereof. In some embodiments, the effective amount is administered once a day to the subject in need thereof over a period of 2 days, 3 days, 4 days, 5 days, 6 days, 7 days, 8 days, 9 days, 10 days, 11 days, 12 days, 13 days, 14 days or more. In some embodiments, the effective amount is administered once a day to the subject in need thereof over a period of up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days. In some embodiments, the effective amount is administered once a day to the subject in need thereof over a period of 7 days. In some embodiments, the effective amount is administered two, three or more times a day to the subject in need thereof. In some embodiments, the effective amount is administered two, three or more times a day to the subject in need thereof over a period of 2 days, 3 days, 4 days, 5 days, 6 days, 7 days, 8 days, 9 days, 10 days, 11 days, 12 days, 13 days, 14 days or more. In some embodiments, the effective amount is administered two, three or more times a day to the subject in need thereof over a period of up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days. In some embodiments, the effective amount is administered two, three or more times a day to the subject in need thereof over a period of 7 days.

In some embodiments, a total dose of about 5 to 100 mg/kg of the chimeric proteins described herein is administered over a period of 4 days, 5 days, 6 days, or 7 days. In some embodiments, a total dose of about 5 to 100 mg/kg of the chimeric proteins described herein is administered over a period of 7 days. In some embodiments, a total dose of about 5 to 90 mg/kg of the chimeric proteins described herein is administered over a period of 4 days, 5 days, 6 days, or 7 days. In some embodiments, a total dose of about 5 to 90 mg/kg of the chimeric proteins described herein is administered over a period of 7 days. In some embodiments, a total dose of about 5 to 80 mg/kg of the chimeric proteins described herein is administered over a period of 4 days, 5 days, 6 days, or 7 days. In some embodiments, a total dose of about 5 to 80 mg/kg of the chimeric proteins described herein is administered over a period of 7 days. In some embodiments, a total dose of about 5 to 70 mg/kg of the chimeric proteins described herein is administered over a period of 4 days, 5 days, 6 days, or 7 days. In some embodiments, a total dose of about 5 to 70 mg/kg of the chimeric proteins described herein is administered over a period of 7 days. In some embodiments, a total dose of about 5 to 60 mg/kg of the chimeric proteins described herein is administered over a period of 4 days, 5 days, 6 days, or 7 days. In some embodiments, a total dose of about 5 to 60 mg/kg of the chimeric proteins described herein is administered over a period of 7 days. In some embodiments, a total dose of about 5 to 50 mg/kg of the chimeric proteins described herein is administered over a period of 4 days, 5 days, 6 days, or 7 days. In some embodiments a total dose of about 5 to 50 mg/kg of the chimeric proteins described herein is administered over a period of 7 days. In some embodiments, a total dose of about 5 to 40 mg/kg of the chimeric proteins described herein is administered over a period of 4 days, 5 days, 6 days, or 7 days. In some embodiments, a total dose of about 5 to 40 mg/kg of the chimeric proteins described herein is administered over a period of 7 days. In some embodiments, a total dose of about 5 to 30 mg/kg of the chimeric proteins described herein is administered over a period of 4 days, 5 days, 6 days, or 7 days. In some embodiments, a total dose of about 5 to 30 mg/kg of the chimeric proteins described herein is administered over a period of 7 days. In some embodiments, a total dose of about 5 to 20 mg/kg of the chimeric proteins described herein is administered over a period of 4 days, 5 days, 6 days, or 7 days. In some embodiments, a total dose of about 5 to 20 mg/kg of the chimeric proteins described herein is administered over a period of 7 days. In some embodiments, a total dose of about 5 to 20 mg/kg of the chimeric proteins described herein is administered over a period of 4 days.

In some embodiments, a total dose of about 100 to 500 mg/kg of the chimeric proteins described herein is administered over a period of 4 days, 5 days, 6 days, or 7 days. In some embodiments, a total dose of about 100 to 500 mg/kg of the chimeric proteins described herein is administered over a period of 7 days. In some embodiments, a total dose of about 100 to 400 mg/kg of the chimeric proteins described herein is administered over a period of 4 days, 5 days, 6 days, or 7 days. In some embodiments a total dose of about 100 to 400 mg/kg of the chimeric proteins described herein is administered over a period of 7 days. In some embodiments, a total dose of about 100 to 300 mg/kg of the chimeric proteins described herein is administered over a period of 4 days, 5 days, 6 days, or 7 days. In some embodiments, a total dose of about 100 to 300 mg/kg of the chimeric proteins described herein is administered over a period of 7 days. In some embodiments, a total dose of about 100 to 200 mg/kg of the chimeric proteins described herein is administered over a period of 4 days, 5 days, 6 days, or 7 days. In some embodiments, a total dose of about 100 to 200 mg/kg of the chimeric proteins described herein is administered over a period of 7 days. In some embodiments, a total dose of about 100 to 150 mg/kg of the chimeric proteins described herein is administered over a period of 4 days, 5 days, 6 days, or 7 days. In some embodiments, a total dose of about 100 to 150 mg/kg of the chimeric proteins described herein is administered over a period of 7 days. In some embodiments, a total dose of about 140 mg/kg of the chimeric proteins described herein is administered over a period of 4 days, 5 days, 6 days, or 7 days. In some embodiments, a total dose of about 140 mg/kg of the chimeric proteins described herein is administered over a period of 7 days.

In some embodiments, the treatment regimen comprises a descending dosage regimen. In a descending dosage regimen, the amount of the dose of the chimeric protein of the disclosure administered in the subject is reduced over the course of the duration of the treatment. In some embodiments, the dose administered on the second day is lower than the dose administered the first day. In other embodiments, the dose administered on the second day is the same than the dose administered the first day, and the dose administered the third day is lower than the dose administered the second day. In some embodiments, the descending dose of chimeric protein is designed to reflect the kinetic mechanism of injury progression and target presentation over time. The amount of phosphatidylserine (PS) target available for chimeric protein binding will be highest during the early stages of acute injury, before the first administration of chimeric protein. Following the initial phase of injury, a smaller amount of injured tissue will be undergoing apoptosis and presenting PS. Previously dosed chimeric protein, provided it has a sufficient half-life, will remain in the tissue in addition to in circulation, engaging with and reducing the amount of available PS for additional chimeric protein to bind to. As the amount of available binding sites decreases, the dose needed to effectively target those sites can descend. In some embodiments, the descending dose also allows for a tapered exposure to chimeric protein, which could limit unwanted side effects or off-target pharmacology.

In some embodiments, the treatment regimen provides for a total dose of about 2 mg/kg to about 200 mg/kg, of about 2 mg/kg to about 20 mg/kg, of about 2 mg/kg to about 10 mg/kg, of about 100 mg/kg to about 200 mg/kg, of about 100 mg/kg to about 150 mg/kg over a predetermined period of time (e.g. 4, 5, 6, or 7 days). In some embodiments, the treatment regimen provides for a total dose of about 5 mg/kg, of about 10 mg/kg, or of about 20 mg/kg over a predetermined period of time (e.g. 4, 5, 6, or 7 days).

In some embodiments, a first daily dose is administered the first day, a second daily dose that comprises from about 85% to about 95% of the amount of the chimeric protein present in the first dose is administered on day 2, a third daily dose that comprises from about 65% to about 85% of the amount of the chimeric protein present in the second dose and an amount that is lower than the second dose is administered on day 3, a fourth dose that comprises from about 45% to about 65% of the amount of the chimeric protein present in the first dose and an amount that lower than the third dose is administered on day 4, a fifth dose that comprises from about 35% to about 45% of the amount of the chimeric protein present in the first dose and an amount that lower than the fourth dose is administered on day 5, a sixth dose that comprises from about 25% to about 35% of the amount of the chimeric protein present in the first dose and an amount that lower than the fifth dose is administered on day 6, and a seventh dose that comprises from about 15% to about 25% of the amount of the chimeric protein present in the first dose and an amount that lower than the sixth dose is administered on day 7. In some embodiments, the descending treatment regimen comprises administering a first daily dose on day 1, a second daily dose corresponding to about 90% of the first daily dose on day 2, a third daily dose corresponding to about 70% of the first daily dose on day 3, a fourth daily dose corresponding to about 50% of the first daily dose on day 4, a fifth daily dose corresponding to about 40% of the first daily dose on day 5, a sixth daily dose corresponding to about 30% of the first daily dose on day 6 and a seventh daily dose corresponding to about 20% of the first daily dose on day 7.

In some embodiments, a first daily dose is administered the first day, a second daily dose that comprises from about 85% to about 95% (e.g. about 90%) of the amount of the chimeric protein present in the first dose is administered on day 2, a third daily dose that comprises from about 65% to about 85% (e.g. about 70%) of the amount of the chimeric protein present in the second dose and an amount that is lower than the second dose is administered on day 3, a fourth dose that comprises from about 45% to about 65% (e.g. about 50%) of the amount of the chimeric protein present in the first dose and an amount that lower than the third dose is administered on day 4.

In some embodiments, a first daily dose administered the first day and the second daily dose administered on the second day are the same, a third daily dose comprising from about 65% to about 90% (e.g. about 75%) of the amount of the chimeric protein present in the first dose is administered on day 3, a fourth daily dose that comprises from about 45% to about 65% (e.g. about 50%) of the amount of the chimeric protein present in the second dose and an amount that is lower than the second dose is administered on day 4.

In some embodiments, the treatment regimen or descending treatment regimen provides a total dose of from about 2 mg/kg to about 20 mg/kg over a period of 7 days. In some embodiments, the treatment regimen provides a total dose of from about 2 mg/kg to about 20 mg/kg over a period of 4 days. In some embodiments, the treatment regimen or descending treatment regimen provides a total dose of from about 2 mg/kg to about 10 mg/kg over a period of 7 days. In some embodiments, the treatment regimen or descending treatment regimen provides a total dose of from about 2 mg/kg to about 10 mg/kg over a period of 4 days. In some embodiments, the treatment regimen or descending treatment regimen provides a total dose of about 5 mg/kg, about 10 mg/kg, or about 20 mg/kg over a period of 7 days. In some embodiments, the treatment regimen or descending treatment regimen provides a total dose of about 5 mg/kg, about 10 mg/kg, or about 20 mg/kg over a period of 4 days.

In some embodiments, the treatment regimen comprises a 5 day course of intravenous administration of the chimeric protein, such as scp776. In some embodiments, the treatment regimen comprises a 7 day course of intravenous administration of the chimeric protein, such as scp776. In some embodiments, the treatment regimen comprises a 4 day course of intravenous administration of the chimeric protein, such as scp776. In some embodiments, the treatment regimen comprises a 3 day course of intravenous administration of the chimeric protein, such as scp776. In some embodiments, the treatment regimen comprises a 2 day course of intravenous administration of the chimeric protein, such as scp776. In some embodiments, the treatment regimen comprises administering intravenously a first dose at from about 2 mg/kg to about 6 mg/kg on day 1, and one dose of about 1 mg/kg to about 2 mg/kg one each of the following days. In some embodiments, the first and second dose are the same (e.g. 2 mg/kg), and the third dose is lower than the first dose. For example, the first dose can be 2 mg/kg, the second dose can be 2 mg/kg, the third dose can be 1.5 mg/kg and the fourth dose can be 1 mg/kg, in a 4 day treatment course. In some embodiments, the second dose is lower than the first dose and the third dose is lower than the second dose etc. . . . . For example, the first dose can be 2 mg/kg, the second dose can be 1.8 mg/kg, the third dose can be 1.4 mg/kg and the fourth dose can be 1 mg/kg, in a 4 day treatment course. In some embodiments, the second dose is lower than the first dose and the third dose is the same as the second dose, etc. . . . . For example, the course can comprise a first dose of about 5.2 mg/kg on day 1, and one dose of about 1.3 mg/kg on day 2, days 3, day 4 and day 5, if the course is a 5 day course.

In some embodiments, the course of treatment comprises administration of the effective amount over 2 days, 3 days, 4 days, 5 days, 6 days, 7 days, 8 days, 9 days, 10 days, 11 days, 12 days, 13 days, 14 days or more. In some embodiments, the course of treatment comprises administration of the effective amount over 7 days. In some embodiments, the course of treatment comprises administration of the effective amount over 4 days. In some embodiments, the course of treatment comprises administration of the effective amount over consecutive days. In some embodiments, the course of treatment comprises administration of the effective amount every day, every 2nd day, every 3rd day or every 4th day.

In some embodiments, the subject in need thereof treated with the chimeric protein or pharmaceutical compositions described herein is continuously infused with dextrose solution to sustain euglycemia. In some embodiments, the dextrose solution comprises from about 5% (w/v) dextrose to about 50% (w/v) dextrose. In some embodiments, the dextrose solution is in sterile water or normal saline solution. For example, the subject in need thereof can be administered i.e., 5% Dextrose in normal saline or 10% Dextrose in sterile water to sustain euglycemia. In some embodiments, the subject in need thereof is administered the chimeric protein or pharmaceutical compositions described herein via bolus injection or intrathecally and is administered a dextrose solution by continuous infusion. In some embodiments, the subject is infused with the dextrose solution for 48 hours. In some embodiments, the subject is infused with the dextrose solution up to the end of the treatment with the chimeric protein. In some embodiments, the subject is infused with the dextrose solution up to 24 hours after the end of the treatment with the chimeric protein. For example, the subject can be treated for 4 days with the pharmaceutical composition described herein and the subject can be infused for up to 4 or 5 days with the dextrose solution.

In some embodiments, the human dose regimen can be calculated based on allometric scaling of the Non-Human Primate to Human dose regimen.

An estimate of the human equivalent dose (HED) to the efficacious dosing regimen can be obtained via allometric scaling (USDHHS, FDA, CDER, Guidance for Industry, 2005). Allometric scaling treats the problem of differential metabolic rates between species by applying a correction factor based on body surface area to the dose of interest in a given species. Allometric scaling is most frequently used in estimating safe starting doses for first-in-human studies, but it is also commonly applied in translating effective doses from animals to man. The FDA-recommended correction factor for converting dosages in 3 kg rhesus monkeys to humans is 3.1 (USDHHS, FDA, CDER, Guidance for Industry, 2005). In some embodiments, the estimated HED of the efficacious dose regimen comprises an initial dose of 16 mg/kg÷3.1=5.2 mg/kg, followed by additional doses at 24 hour intervals of 4 mg/kg÷3.1=1.3 mg/kg.

In some embodiments, the pharmaceutical compositions described herein can further include one or more additional bioactive or therapeutic agents or components to aid in the treatment of damaged tissue or cells and/or facilitate the tissue regenerative process. In some embodiments, the additional therapeutics agent comprises a neuroprotective agent. In some embodiments, the additional therapeutic agent comprises or consists of Glucocorticoids, FK506, Cyclosporin, Sirolimus, nerinetide, oxygen carrying therapeutics (perfluorocarbons) or the like or combination thereof. In some embodiments, the additional therapeutic agent comprises an antibody that targets aggregated forms of amyloid beta. In some embodiments, the additional therapeutic agent comprises an amyloid beta-directed monoclonal antibody. In some embodiments, the additional therapeutic agent comprises or consists of aducanumab, lecanemab or combination thereof.

In some embodiments, the method comprises administering two or more (e.g. two, there or more) pharmaceutical compositions. The different pharmaceutical compositions may be administered to the subject in any order and in any suitable interval. For example, in some embodiments, the one or more some embodiments compositions are administered simultaneously or near simultaneously. In some embodiments, the method comprises a staggered administration of the two or more some embodiments compositions, where a first some embodiments composition is administered and a second some embodiments composition administered at some later time point. Any suitable interval of administration which produces the desired therapeutic effect may be used.

In certain embodiments, the method has an additive effect, wherein the overall effect of the administering a combination of therapeutic agents or procedures is approximately equal to the sum of the effects of administering each therapeutic agent or procedure alone. In other embodiments, the method has a synergistic effect, wherein the overall effect of administering a combination of therapeutic agents or procedures is greater than the sum of the effects of administering each therapeutic agent or procedure alone.

In some embodiments, the pharmaceutical compositions and chimeric proteins described herein, when administered to a subject having a neurodegenerative disease, result in improvement in motor functions and decrease in disease symptoms, such as decrease in tremors in subjects having PD, decreases in autonomic dysfunction in subjects having ALS, or decrease in AD symptoms such as memory loss and confusion in subjects having AD.

In some embodiments, a therapeutically effective amount of the pharmaceutical composition comprising the chimeric protein reduces at least one symptom associated with a nervous system disorder by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein reduces at least one symptom associated with a nervous system disorder by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein disclosed herein reduces at least one symptom associated with a nervous system disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In some embodiments, a therapeutically effective amount of the chimeric protein reduces at least one symptom associated with a nervous system disorder for, e.g., at least one week, at least one month, at least two months, at least three months, at least four months, at least five months, at least six months, at least seven months, at least eight months, at least nine months, at least ten months, at least eleven months, or at least twelve months.

In some embodiments, a therapeutically effective amount of the pharmaceutical composition comprising the chimeric protein results in an improvement of at least one neurological or cognitive function by, e.g., at least 10%, at least 20%, at least 30%, at least 40%, at least 50%, at least 60%, at least 70%, at least 80%, at least 90% or at least 99% or about 100%. In some embodiments, a therapeutically effective amount of the chimeric protein results in an improvement of at least one neurological or cognitive function by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein disclosed herein results in an improvement of at least one neurological or cognitive function by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In some embodiments, a therapeutically effective amount of the chimeric protein results in an improvement of at least one neurological or cognitive function for, e.g., at least one week, at least one month, at least two months, at least three months, at least four months, at least five months, at least six months, at least seven months, at least eight months, at least nine months, at least ten months, at least eleven months, or at least twelve months.

In some embodiments, administration of the effective amount to a subject having an acute CNS injury results in a significant improvement in neurologic function as assessed by a decrease in the Neurologic Deficit Scoring (NDS) such as musculoskeletal coordination, consciousness, motor system; or improvement as assessed by any other neurologic function scale known in the art (e.g. NIH stroke scale, modified Rankin Scale, modified Barthel Index, or Glasgow coma scale, MDS-Unified Parkinson's Disease Rating Scale, or Hunt-Hess Classification).

In some embodiments, administration of the effective amount to a subject having a neurodegenerative disease results in a significant improvement in cognitive function as assessed by, for example, but not limited to, USPDRS, SCAG, and ALS FRS.

In some embodiments, the method of treating neurodegenerative disorders comprises administering a therapeutically effective amount of a chimeric protein to a subject in need thereof. In some embodiments, the chimeric protein comprises (a) a targeting domain comprising a variant of human Annexin 5 (AnxV) comprising one or more mutations, wherein the one or more mutations consist of a substitution at the position corresponding to C316 and optionally at one or more positions corresponding to R63, K70, K101, E138, D139, N160, and combinations thereof; (b) an activator domain comprising a variant of human insulin-like growth factor IGF-1 comprising one or more mutations, wherein the one or more mutations consist of a substitution at one or more positions corresponding to E3, Y24, Y31, Y60, and combinations thereof, wherein the variant of IGF-1 decreases activation of the IGF-1 receptor relative to the wild-type IGF-1, and (c) a half-life modulator comprising a variant of human serum albumin (HSA) comprising one or more mutations, wherein the one or more mutations consist of a substitution at one or more positions corresponding to C58 and N527, and combinations thereof.

In some embodiments, the variant of IGF-1 decreases activation of the IGF-1 receptor relative to the wild-type IGF-1. In some embodiments, the variant of IGF-1 comprises E3R and Y31A substitutions relative to wild type human IGF-1.

In some embodiments, the variant of human Annexin 5 comprises the amino acids 2-320 corresponding to wild type human Annexin 5 and comprises R63A, K70A, K101A, E138A, D139G, N160A, C316A substitutions relative to wild type human Annexin 5.

In some embodiments, the variant of human serum albumin comprises the amino acids 25-609 corresponding to wild type human serum albumin and comprises C58S and N527Q substitutions relative to wild type human serum albumin.

In some embodiments, the chimeric protein is IGF1(E3R/Y31A)_lk7_HSA25-609(C58S/N527Q)_lk7_AnxV2-320(R63A/K70A/K101A/E138A/D139G/N160A/C316A). In some embodiments, the linker lk7 consists of -Gly-Ser-Gly-Gly-Gly-Ser-Gly (SEQ ID NO: 23).

In some embodiments, the chimeric protein is selectively targeted to cells comprising a target molecule phosphatidylserine and exhibits activation of the IGF-1 receptor at least twice as strong on cells containing the target molecule compared to cells that do not contain the target molecule as measured by phosphorylation of serine/threonine protein kinase B (AKT).

In some embodiments, the subject in need thereof has Parkinson disease. In some embodiments, the subject in need thereof has Alzheimer disease.

In some embodiments, 0.01 mg/kg to 100 mg/kg per dose is administered to the subject in need thereof. In some embodiments, up to 200 mg/kg of the chimeric protein is administered to the subject in need thereof.

### Kits

Aspects of the disclosure relates to unit doses and kits. In some embodiments, the kit comprises a supply of individual unit doses comprising the chimeric protein; and instructions for use. In some embodiments, each unit dose is supplied in a suitable container or prefilled package, such as syringes, ampoules or vials. In some embodiments, each kit has a sufficient number of containers or unit dose to treat an individual patient for the suitable numbers of days for a course of treatment.

In some embodiments, the ingredients of the kit are supplied either separately or mixed together in unit dose, for example, as a dry lyophilized powder or water free concentrate in a hermetically sealed container such as an ampoule, vial, syringe or sachette indicating the quantity of active agent. Where the active agent(s) are lyophilized, an ampoule of sterile water or saline for injection can be provided so that the active agent(s) may be mixed prior to injection.

In some embodiments, the kit comprises a plurality of suitable containers, such as vials, and instructions for use. The vials may be fitted with a rubber septum to allow syringe entry for withdrawal of the vial contents. In some embodiments, each vial comprises between 1 and 50 ml extractable volume of the liquid formulation or any volume therebetween. In some embodiments, each vial comprises between 1 and 10 ml extractable volume of the liquid formulation. In some embodiments, each vial comprises 5 ml extractable volume of the liquid formulation. In some embodiments, the kit comprises a plurality of vials with different volume of the liquid formulation, wherein one or more of the plurality of vials comprises a different amount of the chimeric protein. In some embodiments, the plurality of containers comprises a liquid formulation comprising the chimeric protein to be administered in a volume ranging from 1 ml to 50 ml, or any volume therebetween. In some embodiments, the solution comprises about 20 mg/ml of chimeric protein. In some embodiments, the plurality of containers comprises a liquid formulation comprising from 20 mg to about 1 g of the chimeric protein to be administered in a volume ranging from 1 ml to 50 ml or any volume therebetween. In some embodiments, the liquid formulation is ready for injection. In some embodiments, the liquid formulation is diluted into standard IV fluids (e.g., normal saline, 5% dextrose in normal saline, or 10% dextrose in water).

In some embodiments, the volume of the vial in the kit is between 1 mL to 50 mL, corresponding to 20 mg to 1 g of chimeric protein, such as scp776.

The kits described herein may further include other materials desirable from a commercial and user standpoint, including other buffers, diluents, filters, needles, syringes, and package inserts with instructions for performing any methods described herein.

In some embodiments, the instructions for use states to intravenously or intraarterially administer a unit dose from the supply once a day from the supply. In some embodiments, the instructions for use states to intravenously or intraarterially administer a unit dose from the supply two, three or more times a day from the supply. In some embodiments, the instructions for use states to intrathecally administer a unit dose from the supply once a day from the supply. In some embodiments, the instructions for use states to intrathecally administer a unit dose from the supply two, three or more times a day from the supply.

In some embodiments, the supply supports a single course of treatment. In some embodiments, the supply comprises 2, 3, 4, 5, 6, 7, 8, 9, 10, 11, 12, 13, 14 or more individual unit dose (e.g. vials). In some embodiments, the supply comprises 8 individual unit dose (e.g. vials) for a four day treatment course to account for variation in weight of patients and for convenience. In some embodiments, the supply comprises 14 individual unit dose (e.g. vials) for a seven days treatment course to account for variation in weight of patients and for convenience.

In some embodiments, the individual unit dose can be contained in one or more package and are optionally labelled “Dose 1”, “Dose 2”, “Dose 3” etc. . . . or “Day 1”, “Day 2”, Day 3″ etc. . . . .

In some embodiments, the unit dose that is to be administered on day 2 (referred herein as second dose comprises from about 85% to about 95% (e.g. about 90%) of the amount of the chimeric protein present in the unit dose that is to be administered on day 1 (referred herein as first dose), the unit dose that is to be administered on day 3 (referred herein as third dose) comprises from about 65% to about 85% (e.g. about 70%) of the amount of the chimeric protein present in the second dose and an amount that is lower than the second dose, the unit dose that is to be administered on day 4 comprises from about 45% to about 65% (e.g. about 50%) of the amount of the chimeric protein present in the first dose and an amount that lower than the third dose, the unit dose that is to be administered on day 5 comprises from about 35% to about 45% (e.g. about 40%) of the amount of the chimeric protein present in the first dose and an amount that lower than the fourth dose, the unit dose that is to be administered on day 6 comprises from about 25% to about 35% (e.g. about 30%) of the amount of the chimeric protein present in the first dose and an amount that lower than the fifth dose, and the unit dose that is to be administered on day 7 comprises from about 15% to about 25% (e.g. about 20%) of the amount of the chimeric protein present in the first dose and an amount that lower than the sixth dose

In some embodiments, the kit comprises a total dose of about 5 to 100 mg/kg of the chimeric proteins described herein for administration over a period of 4 days, 5 days, 6 days, or 7 days. In some embodiments, the kit comprises a total dose of about 5 to 100 mg/kg of the chimeric proteins described herein for administration over a period of 7 days. In some embodiments, the kit comprises a total dose of about 5 to 90 mg/kg of the chimeric proteins described herein for administration over a period of 4 days, 5 days, 6 days, or 7 days. In some embodiments, the kit comprises a total dose of about 5 to 90 mg/kg of the chimeric proteins described herein for administration over a period of 7 days. In some embodiments, the kit comprises a total dose of about 5 to 80 mg/kg of the chimeric proteins described herein for administration over a period of 4 days, 5 days, 6 days, or 7 days. In some embodiments, the kit comprises a total dose of about 5 to 80 mg/kg of the chimeric proteins described herein for administration over a period of 7 days. In some embodiments, the kit comprises a total dose of about 5 to 70 mg/kg of the chimeric proteins described herein for administration over a period of 4 days, 5 days, 6 days, or 7 days. In some embodiments, the kit comprises a total dose of about 5 to 70 mg/kg of the chimeric proteins described herein for administration over a period of 7 days. In some embodiments, the kit comprises a total dose of about 5 to 60 mg/kg of the chimeric proteins described herein for administration over a period of 4 days, 5 days, 6 days, or 7 days. In some embodiments, the kit comprises a total dose of about 5 to 60 mg/kg of the chimeric proteins described herein for administration over a period of 7 days. In some embodiments, the kit comprises a total dose of about 5 to 50 mg/kg of the chimeric proteins described herein for administration over a period of 4 days, 5 days, 6 days, or 7 days. In some embodiments, the kit comprises a total dose of about 5 to 50 mg/kg of the chimeric proteins described herein for administration over a period of 7 days. In some embodiments, the kit comprises a total dose of about 5 to 40 mg/kg of the chimeric proteins described herein for administration over a period of 4 days, 5 days, 6 days, or 7 days. In some embodiments, the kit comprises a total dose of about 5 to 40 mg/kg of the chimeric proteins described herein for administration over a period of 7 days. In some embodiments, the kit comprises a total dose of about 5 to 30 mg/kg of the chimeric proteins described herein for administration over a period of 4 days, 5 days, 6 days, or 7 days. In some embodiments, the kit comprises a total dose of about 5 to 30 mg/kg of the chimeric proteins described herein for administration over a period of 7 days. In some embodiments, the kit comprises a total dose of about 5 to 20 mg/kg of the chimeric proteins described herein for administration over a period of 4 days, 5 days, 6 days, or 7 days. In some embodiments, the kit comprises a total dose of about 5 to 20 mg/kg of the chimeric proteins described herein for administration over a period of 7 days. In some embodiments, the kit comprises a total dose of about 5 to 10 mg/kg of the chimeric proteins described herein for administration over a period of 4 days, 5 days, 6 days, or 7 days. In some embodiments, the kit comprises a total dose of about 5 to 10 mg/kg of the chimeric proteins described herein for administration over a period of 7 days. In some embodiments, the kit comprises a total dose of about 5 to 10 mg/kg of the chimeric proteins described herein for administration over a period of 4 days. In some embodiments, the kit comprises a total dose of about 10 mg/kg of the chimeric proteins described herein for administration over a period of 4 days, 5 days, 6 days, or 7 days. In some embodiments, the kit comprises a total dose of about 10 mg/kg of the chimeric proteins described herein for administration over a period of 4 days. In some embodiments, the kit comprises a total dose of about 10 mg/kg of the chimeric proteins described herein for administration over a period of 7 days. In some embodiments, the kit comprises a total dose of about 5 mg/kg of the chimeric proteins described herein for administration over a period of 4 days, 5 days, 6 days, or 7 days. In some embodiments, the kit comprises a total dose of about 5 mg/kg of the chimeric proteins described herein for administration over a period of 7 days. In some embodiments, the kit comprises a total dose of about 5 mg/kg of the chimeric proteins described herein for administration over a period of 4 days.

In some embodiments, the kit comprises a total dose of about 100 to 500 mg/kg of the chimeric proteins described herein for administration over a period of 4 days, 5 days, 6 days, or 7 days. In some embodiments, the kit comprises a total dose of about 100 to 500 mg/kg of the chimeric proteins described herein for administration over a period of 7 days. In some embodiments, the kit comprises a total dose of about 100 to 400 mg/kg of the chimeric proteins described herein for administration over a period of 4 days, 5 days, 6 days, or 7 days. In some embodiments, the kit comprises a total dose of about 100 to 400 mg/kg of the chimeric proteins described herein for administration over a period of 7 days. In some embodiments, the kit comprises a total dose of about 100 to 300 mg/kg of the chimeric proteins described herein for administration over a period of 4 days, 5 days, 6 days, or 7 days. In some embodiments, the kit comprises a total dose of about 100 to 300 mg/kg of the chimeric proteins described herein for administration over a period of 7 days. In some embodiments, the kit comprises a total dose of about 100 to 200 mg/kg of the chimeric proteins described herein for administration over a period of 4 days, 5 days, 6 days, or 7 days. In some embodiments the kit comprises a total dose of about 100 to 200 mg/kg of the chimeric proteins described herein for administration over a period of 7 days. In some embodiments, the kit comprises a total dose of about 100 to 150 mg/kg of the chimeric proteins described herein for administration over a period of 4 days, 5 days, 6 days, or 7 days. In some embodiments, the kit comprises a total dose of about 100 to 150 mg/kg of the chimeric proteins described herein for administration over a period of 7 days. In some embodiments, the kit comprises a total dose of about 140 mg/kg of the chimeric proteins described herein for administration over a period of 4 days, 5 days, 6 days, or 7 days. In some embodiments, the kit comprises a total dose of about 140 mg/kg of the chimeric proteins described herein for administration over a period of 7 days.

In some embodiments, the instructions for use comprises dosing schedule treatment in a readable medium such as pamphlet or written information. In some embodiments, the readable medium comprises specifications of the active agents, preparation instructions, dosage variations, storage instructions, administration instructions, side effects of the pharmaceutical ingredients, contraindications for administration, stability data or any other information relevant to the use of the composition described herein for patient care or physician information.

Aspects of the disclosure relate to the use of pharmaceutical composition for the treatment of neurovegetative disease, the pharmaceutical composition comprising a therapeutically effective amount of a chimeric protein described herein and a pharmaceutically acceptable carrier, wherein the pharmaceutical composition is administered in the form of bolus injection. Other aspects of the disclosure relate to the use of pharmaceutical composition for the treatment of neurovegetative disease, the pharmaceutical composition comprising a therapeutically effective amount of a chimeric protein described herein and a pharmaceutically acceptable carrier, wherein the pharmaceutical composition is administered in the form of intrathecal injection. In some embodiments, the chimeric protein comprises (a) a targeting domain comprising a variant of human Annexin 5 (AnxV) comprising one or more mutations, wherein the one or more mutations consist of a substitution at the position corresponding to C316 and optionally at one or more positions corresponding to R63, K70, K101, E138, D139, N160, and combinations thereof; (b) an activator domain comprising a variant of human insulin-like growth factor IGF-1 comprising one or more mutations, wherein the one or more mutations consist of a substitution at one or more positions corresponding to E3, Y24, Y31, Y60, and combinations thereof, wherein the variant of IGF-1 decreases activation of the IGF-1 receptor relative to the wild-type IGF-1, and (c) a half-life modulator comprising a variant of human serum albumin (HSA) comprising one or more mutations, wherein the one or more mutations consist of a substitution at one or more positions corresponding to C58 and N527, and combinations thereof.

### EXAMPLES

The following Examples are offered by way of illustration and not by way of limitation. Unless otherwise specified, all reagents and solvents are of standard commercial grade and are used without further purification. Using routine modifications, the procedures provided in the following Examples may be varied by those of ordinary skill in the art to make and use other bi-specific fusion proteins and pharmaceutical compositions within the scope of the present disclosure.

### Example 1: Efficacy Study of Scp776 in a Non-Human Primate Model (NHP) of Ischemic Stroke

### Methods

1. Description of the Transient Middle Cerebral Artery Occlusion (tMCAO) Ischemic Stroke (IS) Model in Cynomolgus Fascicularis (Macaca fascicularis)

Under anesthesia, the right eye globe was removed. Under an operating microscope (Kom300, Konan Medical, Hogo, Japan), the orbital content was dissected and excised. A window of approximately 10 mm in diameter was opened just anterior to the foramen of the optic nerve at the base of the skull. The main trunk of the right middle cerebral artery (MCA) was visible through the window, which is beneath dura matter. After opening the dura matter, tMCAO was performed using two microvascular clips (Mini #81, Sugita Aneurysm Clips, Mizuho Medical Corp., Tokyo, Japan), one on the proximal part of the main MCA trunk and the other on the distal-to-orbitofrontal branch. These clips were removed four (4) hours after MCA occlusion. After visual confirmation of re-establishment of MCA blood flow, the burr hole was closed using Clearfil New Bond (Kuraray Noritake Dental, Inc., Tokyo, Japan) and the incision was sutured. A pair of new clips was used for each macaque.

Any abnormal observations during clip placement and/or removal, such as vessel rupture or bleeding, was noted and reported.

### 2. Neurologic Deficit Scoring (NDS) Method

Twenty-four (24) hours (±2 hrs.), 48 hrs. (±2 hrs.), 72 hrs. (±2 hrs.), 7, 10 and 14 days after occlusion, neurologic deficits were obtained according to the method described by Kito et al. (J. Neurosci. Meth., 2001; 105: 45-53).

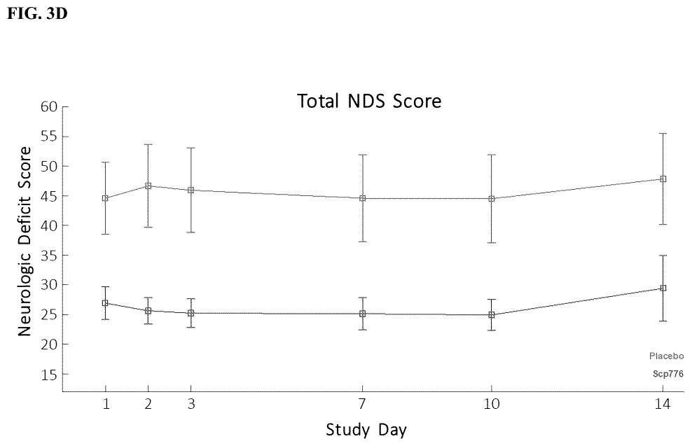

FIG. 3A shows the NDS motor system score at 48 hours (day 2) of placebo treated and scp776 treated animals.

FIG. 3B shows the NDS consciousness score at day 7 of placebo treated and scp776 treated animals.

FIG. 3C shows the NDS musculoskeletal coordination score at day 1, 2, 3, 7, 10, and 14 of placebo treated and scp776 treated animals.

FIG. 3D shows the total NDS score at day 1, 2, 3, 7, 10, and 14 of placebo treated and scp776 treated animals.

### 3. Magnetic Resonance Imaging (MRI) Method

Brain images using MRI were obtained 4 hrs. after reperfusion (i.e. 8 hrs. after occlusion), 72 hrs. (±2 hrs.), and 14 days after occlusion.

During imaging, macaques were anesthetized with propofol (12-20 mg/kg; Maruishi Pharmaceutical Co., Osaka, Japan). Animals were fixed on a MRI bed (Signa Explorer 1.5T, GE Healthcare, Milwaukee, WI), and serial coronal images (6 mm thickness, vertical plane against orbitomeatal line) were be obtained. All imaging sequences consisted of the following:
Diffusion-weighted imaging (DWI);
Arterial spin labeling (ASL);
Apparent diffusion coefficient (ADC);
T1;
Fluid-attenuated inversion-recovery (FLAIR); and
T2-weighted imaging.

For the MRI conducted at the 8 hr. time point, in order to visualize blood brain barrier (BBB) disruption following treatment, contrast-enhanced T1-weighted imaging consisted of T1-weighted whole brain volume acquired before and about 5 min. after intravenous injection of gadobutrol contrast agent (1.0 mmol/mL, 0.2 mL/kg; Gadovist; Bayer HealthCare Pharmaceuticals, Osaka, Japan). All animals received a dose volume of 0.2 mL/kg.

Apparent diffusion coefficient (ADC) maps, cerebral blood flow (CBF), and perfusion deficit was generated with software (READY View, GE Healthcare) available on the MR scanner console for all MRIs done in the study.

The lesion volume (mm³) was calculated as the sum of the infarct area of each section and thickness (6 mm). Lesion volumes were calculated in cerebral cortex, white matter and basal ganglia and the total lesion volume was the sum of these volumes.

The area of BBB disruption (mm²) was delineated from each contrast-enhanced T1-weighted image using OsiriX MD version 12.0.0. BBB disruption was manually delineate using trace contrast-enhanced T1-weighted images. The volume (mm³) of BBB disruption was calculated as the sum of the areas of BBB disruption of each section (6 mm thickness).

### Dosage

All animals received one (1) dose of 1 ml/kg thirty minutes prior to release of the clips (or 3.5 hours after clip placement) and an additional four doses separated by 24 hours for a total of five (5) doses via Intravenous (i.v.) slow push over approximately 30 seconds. Placebo corresponds to vehicle alone (20 mM Tris, 7.5% w/v Sucrose, 0.02% w/v polysorbate 80, pH 7.5).

All animals received a total of five (5) doses. The dosage regimen is shown the table below. The dose 16 mg/kg corresponds to 16 mg/ml and the dose 4 mg/kg correspond to 4 mg/ml of the chimeric protein. The interval between administrations was no less than 20 hours and no more than 28 hours. Any instances where the interval between administrations falls outside of this range (20-28 hours) was reported.

| | | | | | | |
|---|---|---|---|---|---|---|
| Placebo | Day: | 1 | 2 | 3 | 4 | 5 |
| (n = 12) | Treatment: | placebo | placebo | placebo | placebo | placebo |
| Divided | Treatment: | 16 mg/kg | 4 mg/kg | 4 mg/kg | 4 mg/kg | 4 mg/kg |
| Dose | | scp776 | scp776 | scp776 | scp776 | scp776 |
| (n = 10) | | | | | | |

### Dextrose Supplementation

In some embodiments, the animals being treated are continuously infused at a rate of 0.5 mL/kg/hr with dextrose to sustain euglycemia. Dextrose solution can comprise between about 5% to about 50% (w/v) dextrose in sterile water or saline solution.

### NDS, MRI, and Survival Results Summary

Animals were assessed using NDS scoring: The four systems for the NDS scoring system are Motor, Consciousness, Musculoskeletal coordination, and Sensory. The Total NDS score is the sum of the four systems.

Treatment with scp776 significantly improves neurologic function in three of the four systems that are assessed in the NDS scoring when compared with treatment with placebo.
Significant improvements to Consciousness were observed at day 3 (not shown) and day 7 (see FIG. 3B).
Significant improvements to Motor System were observed at day 2 (see FIG. 3A).
Significant improvements to Musculoskeletal Coordination were observed at day 1, 2, 3, 7, 10, and 14 (See FIG. 3C).
Improvement to total NDS score were observed at all time points. Significant improvements to total NDS score were observed at day 1 and day 3 (see FIG. 3D).

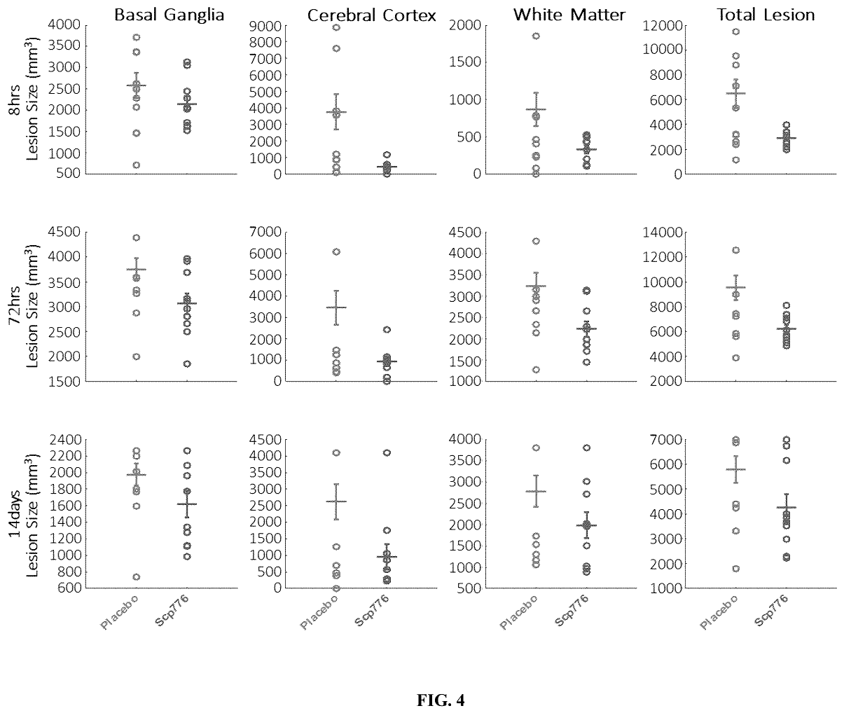

At all time points evaluated, treatment with scp776 resulted in a smaller lesion size or area than treatment with placebo (see FIG. 4).
The reduction in lesion volume achieved statistical significance in the cerebral cortex and total lesion volumes at 8 hrs.
Total lesion volume was significantly reduced at 72 hours by scp776 treatment.
The volume of lesion in the cerebral cortex was significantly reduced by scp776 treatment at 14 days.

Scp776 treatment resulted in a statistically significant survival benefit. In the placebo group, 50% of the animals died as a result of the stroke injury, while only 10% of animals that received scp776 died prior to study completion. This survival difference, when assessed by the log rank method and chi-squared distribution, reveals a p-value of 0.039 (Bewick et al. Critical Care 2004, 8:389-394).

### Example 2. Treatment of Parkinson's Disease (PD), Amyotrophic Lateral Sclerosis (ALS), and Alzheimer's Disease (AD)

Parkinson's Disease (PD), Amyotrophic lateral sclerosis (ALS), and Alzheimer's Disease (AD) are characterized by progressive degeneration of the central or peripheral nervous systems. Each of these diseases is associated with impaired musculoskeletal coordination and motor system control. The model of Acute Ischemic Stroke in the non-human primates induces significant disfunction in both musculoskeletal coordination and motor control (See example 1). See US Patent application Publication No. 2022-0031812 which is incorporated by reference herein in its entirety. The neuroprotectant activity of scp776 significantly reduces these disfunctions. The neuroprotectant activity of scp776 is independent of the type of insult that results in injured tissue. In chronic neurologic diseases, brain tissue is also injured and undergoes similar distress at the cellular level. Taken together, these results and the mode of disease progression, indicate that if scp776 were to be administered during symptomatic progression, the neuroprotective outcome observed in the model of acute ischemic stroke would translate to the chronic disease setting.

Experiments are conducted to examine the effect of scp776 on the treatment of Parkinson disease. Scp776 is dosed from about 0.01 mg/kg to about 20 mg/kg as often as daily. Administration results in improvements of, for example but not limited to, the measures of motor function and decrease in neurodegenerative symptoms, such as decreases of tremors.

Experiments are conducted to examine the effect of scp776 on the treatment of Amyotrophic lateral sclerosis (ALS). Scp776 is dosed from about 0.01 mg/kg to about 20 mg/kg as often as daily. Administration results in improvements of, for example but not limited to, measures of motor function and decreases on ALS symptoms such as autonomic dysfunction.

Experiments are conducted to examine the effect of scp776 on the treatment of Alzheimer's Disease (AD). Scp776 is dosed from about 0.01 mg/kg to about 20 mg/kg as often as daily. Administration results in improvements of, for example but not limited to, measures of cognitive function and decreases on AD symptoms such as memory loss and confusion.

### Example 3. Treatment of Synucleinopathies

The term α-synucleinopathies is usually employed to define a group of neurodegenerative disorders that show common pathologic proteinaceous accumulation of α-synuclein aggregates which become organized into insoluble, amyloid fibrils. Alpha-synuclein aggregates can deposit in selective populations of neuronal and glial cells. Alpha-synucleinopathies include symptomatically heterogeneous disorders such as Lewy bodies-associated diseases (e.g. Parkinson's Disease (PD), Lewy body dementia, diffuse Lewy body disease, cortical Lewy body disease, and senile dementia of Lewy type, multiple system atrophy, Lewy bodies dysphagia).

Parkinson's disease (PD) is a common neurodegenerative disorder that affects 1% of the population over 65. Parkinson's Disease (PD) is characterized by mis-regulation of alpha-Synuclein, forming aberrant fibrils. These fibrils act as both intra- and extra-cellular toxins, promoting neuronal cell death. They are present in other neurodegenerative conditions, collectively termed synucleinopathies. Alzheimer's Disease (AD) can exhibit characteristics of synucleinopathies and share the pathophysiology of synucleinopathies.

While most therapeutic strategies against PD are focused on designing agents that can directly target α-Syn and the amyloid fibrils, the chimeric proteins disclosed herein slow the formation of fibrils by restoring a natural signaling pathway. It is known that the accumulation of mis-folded alpha-synuclein fibrils inhibits AKT. In some embodiments, the chimeric proteins stimulate the AKT pathway (by activation of the IGF1-Receptor) to override the mis-regulated alpha-synuclein pathway thereby preventing or reversing fibril formation. In some embodiments, the chimeric protein provided herein slow disease progression.

While PD symptomology does indicate a leaky blood brain barrier (BBB), it is likely not as permissive as the BBB of stroke patients. Therefore, in some embodiments, the chimeric protein is administered intrathecally (IT).

Intrathecal and/or intranigral formulations of the chimeric proteins are dosed to animals infected and not infected with a pathogenic variant of the alpha-synuclein gene and rate of clearance from cerebrospinal fluid (CSF) and brain tissue is measured.

### Example 4: Assessment of the Pharmacokinetic/Pharmacodynamic Relationship of Scp776 in the AAV1/2-hA53T-aSyn Rat Model of Parkinson's Disease—Preliminary Phase

The value of current therapies for Parkinson's disease is limited by the fact they do little, if anything, to modify the underlying progression of the disease. A novel therapy, with disease-modifying actions, be they neuroprotective, neurorestorative or compensatory to slow, halt or even reverse disease and/or symptom progression would be of great value in the treatment of Parkinson's disease.

This study provides specimens for analysis and assessment of the pharmacokinetic/pharmacodynamic (PK/PD) relationship of potential disease-modifying scp776 following intrathecal (IT) and intranigral (IN) administration in the AAV1/2-hA53T-aSyn rat model of Parkinson's disease.

### Preliminary Phase:

Test subjects: The preliminary phase incorporated a total of three treatment groups with N=3 rats per group (total N=9, healthy female Sprague-Dawley rats).

Route of administration: A pharmaceutical composition comprising scp776 was administered directly into the substantia nigra (ISN) using stereotaxic techniques.

Dose groups: The dose volume for all test subjects is 8 μL administered by syringe pump at a rate of 0.5 μL/minute.

| | Pharmaceutical composition | | |
|---|---|---|---|
| Group | comprising scp776/Vehicle | Dose | Collection |
| X | Scp776 | 10 μg | +4 h |
| Y | Scp776 | 40 μg | +4 h |
| Z | Scp776 | 160 μg | +4 h |

Formulation: Formulations of scp776 at 1.25 g/1, 5 g/l, and 20 g/l in a buffer containing 20 mM NaCitrate, 122 mM NaCl, pH 6.5 were used.

Human brain is on average ˜1000 times heavier than rat brain: the corresponding human equivalent dosages is about 10, 40, and 160 mg, respectively.

Specimen Collection: Animals were killed 4 hours following completion of administration of the composition comprising scp776. Cerebrospinal fluid (CSF), serum, and brain tissue samples are collected.

Brain tissue samples were fixed in formalin, paraffin embedded, and stained for the presence of scp776 using an immunohistochemistry (IHC) method that is specific for the human serum albumin portion of scp776.

Primary Objective: The purpose of the preliminary phase was to assess tolerability of the selected dose levels when administered via this route.

Secondary Objective: The secondary purpose was to assess the amount of scp776 localization in the substantia nigra (SN) by IHC.

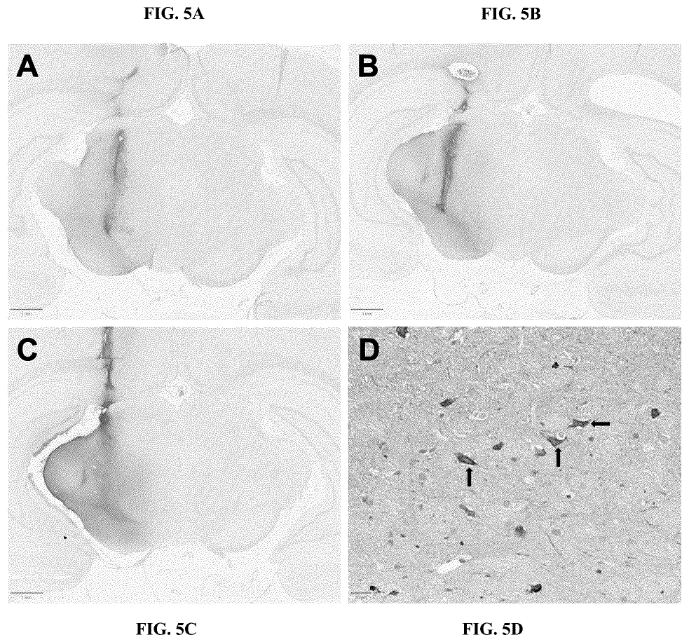

Results and Discussion: Intranigral dosing of scp776 was well tolerated, with no changes in behavior observed following the dose in any animal until takedown at 4 hours post-dose. IHC staining of coronal brain sections for scp776 revealed a dose-dependent and consistent distribution pattern across the animals. As shown in FIGS. 5A-5C, intense staining directly adjacent to the sites of injection was apparent, with more diffuse staining spreading through the anatomical right SN (appears on the left side of the image slice) and surrounding regions. Diffusion of scp776 away from the injection site injury, and potential source of excess phosphatidylserine, and accumulation in the SN of healthy animals was unexpected and should enable a meaningful comparison of scp776 levels that accumulate in healthy versus aSyn pathological tissue in future studies. The apparent persistence of scp776 in brain tissue at 4 hours post-intranigral dose was unexpected, considering the well-established, rapid turnover of CSF and the possibility for active clearance of scp776 by microglia or other mechanisms. Uptake into the cytoplasm of select cells (for example astrocytes based on visual analysis) was observed in most images, indicating potential drug engagement at the cell surface leading to internalization (FIG. 5D).

### Example 5: Assessment of the Pharmacokinetic/Pharmacodynamic Relationship of Scp776 in the AAV1/2-hA53T-aSyn Rat Model of Parkinson's Disease—Main Phase

In previous studies, IT and intranigral injections of up to 160 μg scp776 formulated in 20 mM NaCitrate, 122 mM NaCl, pH 6.5 were well tolerated by healthy rats.

The main phase incorporates a total of eleven treatment groups with N=4 rats per group (total N=44, female Sprague-Dawley rats).

Test subjects: All subjects in the main phase are infected via stereotaxic injection to their right substantia nigra with either the empty vector (EV) AAV or the human pathogenic variant of alpha-synuclein (hA53T-aSyn) AAV.

### Routes of Administration:

ISN: The pharmaceutical composition comprising scp776 is administered directly into the substantia nigra (ISN) using stereotaxic techniques.

IT: The pharmaceutical composition comprising scp776 is administered directly into the cerebrospinal fluid compartment via intrathecal (IT) catheters at the level of the L3-L5 vertebrae.

Dose Groups: The dose volume for all test subjects is 8 μL administered by syringe pump at a rate of 0.5 μL/minute. The dose amount of scp776 is determined in the Preliminary Phase of the study.

| | | Pharmaceutical | | | |
|---|---|---|---|---|---|
| | | composition | | | |
| | | comprising | | | |
| | | scp776/ | ISN/ | | Collec- |
| Group | AAV | Vehicle | IT | Day | tion |
| 1 | EV | Scp776 | ISN | D15 | +1 h |
| 2 | hA53T-aSyn | Vehicle | ISN | D15 | +1 h |
| 3 | hA53T-aSyn | Scp776 | ISN | D15 | +1 h |
| 4 | hA53T-aSyn | Scp776 | ISN | D15 | +4 h |
| 5 | hA53T-aSyn | Scp776 | ISN | D15 | +24 h |
| 6 | hA53T-aSyn | Vehicle | ISN | D29 | +1 h |
| 7 | hA53T-aSyn | Scp776 | ISN | D29 | +1 h |
| 8 | hA53T-aSyn | Scp776 | ISN | D29 | +4 h |
| 9 | hA53T-aSyn | Scp776 | ISN | D29 | +24 h |
| 10 | hA53T-aSyn | Scp776 | IT | D15 | +1 h |
| 11 | hA53T-aSyn | Scp776 | IT | D29 | +1 h |

Primary Objective: The purpose of the main phase is the quantitation of the amount of scp776 localization to the right SN as determined by IHC.

The relative accumulation and retention of scp776 in the portion of the SN that has been infected by the hA53T-aSyn AAV is compared between the two times of administration (i.e., D15 and D29).

Secondary Objective: The secondary purpose of the main phase is the determination of IT administration feasibility for delivery of scp776 to AAV infected SN.

### Exploratory Objectives:

1. Quantification of striatal dopamine levels by LC-MS/MS.
2. Quantification of transgene-derived human alpha synuclein by ELISA.
3. Quantification of neurofilament light chain (NfL) in serum/CSF by ELISA.
4. Microglia activation assessment via Iba-1 immunohistochemical staining.

Interpretation of Results: The aim of the study is to demonstrate a selective accumulation and retention of scp776 in the right substantia nigra of hA53T-aSyn infected rats which will indicate that the targeting functionality of scp776 is preserved in the CSF space and brain tissue.

The tissue half-life for scp776 in the SN can be estimated by comparing the groups with different takedown times (1, 4, or 24 hours).

The kinetics of scp776 target presentation (apoptotic cells exposing phosphatidylserine) in this model can be determined by comparing the groups that were dosed 15 or 29 days post AAV injection and can inform subsequent efficacy studies.

IT bioavailability of scp776 in the context of this model can be estimated by comparing the IT and ISN routes of administration.

The exploratory objectives are efficacy assessments. The directionality of potential therapeutic benefit with administration of pharmaceutical composition comprising scp776 in hA53T-aSyn infected rats is hypothesized to be one or more of the following:
Increase in striatal dopamine
Decrease in serum neurofilament light chain (NfL)
Decrease in microglial activation

Specific examples of compositions, methods and kits have been described herein for purposes of illustration. These are only examples. The technology provided herein can be applied to systems other than the example systems described above. Many alterations, modifications, additions, omissions, and permutations are possible within the practice of this invention. This disclosure includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

### INCORPORATION BY REFERENCE

All publications, patents and sequence database entries mentioned herein are hereby incorporated by reference in their entirety as if each individual publication or patent was specifically and individually indicated to be incorporated by reference.

1. A method of treating neurodegenerative disorder, the method comprising administering to a subject in need thereof a pharmaceutical composition comprising a therapeutically effective amount of a chimeric protein and a pharmaceutically acceptable carrier,
wherein the chimeric protein comprises (a) a targeting domain comprising a variant of human Annexin 5 (AnxV) comprising one or more mutations, wherein the one or more mutations consist of a substitution at the position corresponding to C316 and optionally at one or more positions corresponding to R63, K70, K101, E138, D139, N160, and combinations thereof; (b) an activator domain comprising a variant of human insulin-like growth factor IGF-1 comprising one or more mutations, wherein the one or more mutations consist of a substitution at one or more positions corresponding to E3, Y24, Y31, Y60, and combinations thereof, wherein the variant of IGF-1 decreases activation of the IGF-1 receptor relative to the wild-type IGF-1, and (c) a half-life modulator comprising a variant of human serum albumin (HSA) comprising one or more mutations, wherein the one or more mutations consist of a substitution at one or more positions corresponding to C58 and N527, and combinations thereof.

2. The method of claim 1, wherein administration of the therapeutically effective amount of the chimeric protein reduces or prevent amyloid fibril formation.

3. The method of claim 1, wherein the subject in need thereof has Parkinson disease.

4. The method of claim 1, wherein the subject in need thereof has Alzheimer disease.

5. The method of claim 1, wherein the variant of IGF-1 comprises E3R and Y31A substitutions relative to wild type human IGF-1.

6. The method of claim 1, wherein the variant of human Annexin 5 comprises the amino acids 2-320 corresponding to wild type human Annexin 5 and comprises R63A, K70A, K101A, E138A, D139G, N160A, C316A substitutions relative to wild type human Annexin 5.

7. The method of claim 1, wherein the variant of human serum albumin comprises the amino acids 25-609 corresponding to wild type human serum albumin and comprises C58S and N527Q substitutions relative to wild type human serum albumin.

8. The method of claim 1, wherein the chimeric protein is IGF1(E3R/Y31A)_lk7_HSA25-609(C58S/N527Q)_lk7_AnxV2-320(R63A/K70A/K101A/E138A/D139G/N160A/C316A).

9. The method of claim 6, wherein the linker lk7 comprises -Gly-Ser-Gly-Gly-Gly-Ser-Gly (SEQ ID NO: 23).

10. The method of claim 1, wherein the chimeric protein is selectively targeted to cells comprising a target molecule phosphatidylserine and wherein the chimeric protein exhibits activation of the IGF-1 receptor at least twice as strong on cells containing the target molecule compared to cells that do not contain the target molecule as measured by phosphorylation of serine/threonine protein kinase B (AKT).

11. The method of claim 1 comprising administering daily from about 0.01 mg/kg to about 20 mg/kg of the chimeric protein to the subject in need thereof.

12. The method of claim 1 comprising administering a total dose of from about 5 mg/kg to about 20 mg/kg of the chimeric protein to the subject in need thereof over a period of 4 to 7 days.

13. The method of claim 1, comprising administering descending doses of the chimeric protein to the subject in need thereof.

14. The method of claim 1, comprising administering to the subject in need thereof a first dose comprising from about 2 mg/kg to about 6 mg/kg of the chimeric protein on day 1, and a dose comprising from about 1 mg/kg to about 2 mg/kg one each of the following days.

15. The method of claim 1, wherein the administering comprises administering to the subject in need thereof a first dose of the pharmaceutical composition on day 1 and a second dose of the pharmaceutical composition on day 2, wherein the first dose comprises an amount of chimeric protein that is higher than an amount of chimeric protein in the second dose, and wherein the first dose comprises from about 2 mg/kg to about 6 mg/kg of the chimeric protein.

16. The method of claim 1, comprising administering intravenously, intraarterially, intranigrally, or intrathecally the pharmaceutical composition.

17. The method of claim 1, comprising administering intravenously the pharmaceutical composition.

18. The method of claim 1, comprising administering intrathecally the pharmaceutical composition.

19. A method of treating Parkinson disease, the method comprising administering intrathecally or intranigrally to a subject in need thereof a pharmaceutical composition comprising a therapeutically effective amount of a chimeric protein and a pharmaceutically acceptable carrier,
wherein the chimeric protein comprises (a) a targeting domain comprising a variant of human Annexin 5 (AnxV) comprising one or more mutations, wherein the one or more mutations consist of a substitution at the position corresponding to C316 and optionally at one or more positions corresponding to R63, K70, K101, E138, D139, N160, and combinations thereof; (b) an activator domain comprising a variant of human insulin-like growth factor IGF-1 comprising one or more mutations, wherein the one or more mutations consist of a substitution at one or more positions corresponding to E3, Y24, Y31, Y60, and combinations thereof, wherein the variant of IGF-1 decreases activation of the IGF-1 receptor relative to the wild-type IGF-1, and (c) a half-life modulator comprising a variant of human serum albumin (HSA) comprising one or more mutations, wherein the one or more mutations consist of a substitution at one or more positions corresponding to C58 and N527, and combinations thereof.

20. The method of claim 19, wherein the pharmaceutical composition is free of sucrose.


SEQUENCE LISTING (26 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 70 aa, synthetic construct
  REGION 1..70: note = synthetic construct
GPRTLCGAELVDALQFVCGDRGFYFNKPTGAGSSSRRAPQTGIVDECCFRSCDLRRLEMYCAPLKPAKSA

SEQ ID NO: 3 - protein, 83 aa, synthetic construct
  REGION 1..83: note = synthetic construct
MFPAMPLSSLFVNGPETLCGAELVDALQFVCGDRGFYFNKPTGYGSSSRRAPQTGIVDECCFRSCDLRRLEMYCAPLKPAKSA

SEQ ID NO: 4 - protein, 70 aa, synthetic construct
  REGION 1..70: note = synthetic construct
GPRTLCGAELVDALQFVCGDRGFYFNKPTGYGSSSRRAPQTGIVDECCFRSCDLRRLEMYCAPLKPAKSA

SEQ ID NO: 5 - protein, 67 aa, synthetic construct
  REGION 1..67: note = synthetic construct
TLCGAELVDALQFVCGDRGFYFNKPTGYGSSSRRAPQTGIVDECCFRSCDLRRLEMYCAPLKPAKSA

SEQ ID NO: 6 - protein, 83 aa, synthetic construct
  REGION 1..83: note = synthetic construct
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 7 - protein, 69 aa, synthetic construct
  REGION 1..69: note = synthetic construct
GPETLCGAELVDALQFVCGDRGFYFNKPTGYGSSSRAPQTGIVDECCFRSCDLRRLEMYCAPLKPAKSA

SEQ ID NO: 8 - protein, 67 aa, synthetic construct
  REGION 1..67: note = synthetic construct
GPETLCGAELVDALQFVCGDRGFYFNKPTGYGSSSRRAPQTGIVDECCFRSCDLRRLEMYCAPLKPA

SEQ ID NO: 9 - protein, 319 aa, Homo sapiens
AQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 10 - protein, 319 aa, synthetic construct
  REGION 1..319: note = synthetic construct
AQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLAGEDD

SEQ ID NO: 11 - protein, 319 aa, synthetic construct
  REGION 1..319: note = synthetic construct
AQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 12 - protein, 585 aa, Homo sapiens [sequence omitted]

SEQ ID NO: 13 - protein, 585 aa, synthetic construct
  REGION 1..585: note = synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGL

SEQ ID NO: 14 - protein, 585 aa, synthetic construct
  REGION 1..585: note = synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAGTFTFHADICTLSEKERQIKKQTALVELVKHKPKATKEQLKAAMDDFAAFVEKCCKADDKETCFAEEGKKLVAASQAALGL

SEQ ID NO: 15 - protein, 585 aa, synthetic construct; identical to SEQ ID NO: 13
  REGION 1..585: note = synthetic construct

SEQ ID NO: 16 - protein, 20 aa, synthetic construct
  REGION 1..20: note = synthetic construct
GGGGSGGGGSGGGGSGGGGS

SEQ ID NO: 17 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 18 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 19 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 20 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 21 - protein, 20 aa, synthetic construct
  REGION 1..20: note = synthetic construct
ASPAAPAPASPAAPAPSAPA

SEQ ID NO: 22 - protein, 107 aa, synthetic construct
  REGION 1..107: note = synthetic construct
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIK

SEQ ID NO: 23 - protein, 7 aa, synthetic construct
  REGION 1..7: note = synthetic construct
GSGGGSG

SEQ ID NO: 24 - protein, 988 aa, synthetic construct
  REGION 1..988: note = synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLGSGGGSGAQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLAGEDD

SEQ ID NO: 25 - protein, 988 aa, synthetic construct
  REGION 1..988: note = Synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLGSGGGSGAQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLSGEDD

SEQ ID NO: 26 - protein, 70 aa, synthetic construct
XXXXLCXXXLXXXLXXXCXXXXFXFXXXXXXXXXXXXXXXXXIXXXCCXXXCXLXXLXXXCXXXXXXXXX